SEQUENCE LISTING (40 sequences)

SEQ ID NO: 1 - protein, 2144 aa, Triticum aestivum
MPTAQGDWFEAAAPPGGRRARRSQSASAPCRSARQAHGAMSLDGGFLGGAQRTEERVAPRPPRAAARDAESIRPMSLLPESSIGLYDPAFERDSCGVGFVAELSGVDNRATVVDAIQMLERMAHRGACGCEKNTGDGAGILVALPHTFFREVTKDAGFELPPPGEYAVGMVFLPTDEKRRERSKTEFTKVAESLGHSILGWRQVPTDNSDLGQAALDTEPAIEQVFLTKSSKSKADFEQQVFILRRLSIVSIRAALNLQRGGERDFYMCSLSSRTIVYKGQLMPSQLQGYYYADIGHENFSSYMALVHSRFSTNTFPSWDRAQPMRVLGHNGEINTLKGNKNWMKAREGLLECEKLGLSQDEMSKILPIVDATSSDSGAFDGVLELLIRGGRSLPEAVMMMIPEAWQNDVNMEPDKKALYEFLSALMEPWDGPALISFTDGRYLGATLDRNGLRPGRFYVTHSGRVVMGSEVGVVDIPAQDVLRKGRLNPGMMLLVDFDNHTVVDDEALKAQYSKAHPYGEWLKRQKMYLKDIVESVPETDRVAPSISGSITQTNENKECVGINAIVTPLKAFGYTLEALEMLLLPMAKDGVEALGSMGNDAPLAVMSNREKLTFEYFKQMFAQVTNPPIDPIREKIVTSMECMIGPEGDLLEITEKQCNRLALKGPLVSMDEMESIKKMNYRGWRSKVLDITYPKNSGRKGLEETLDRICAEAREAIREGYKILVLSDRGFSSDRVAVSSLLAVGAVHQHLVANLERTRVGLLVESAEPREVHHFCTLVGFGADAICPYLAIEAIWCLQTDGKIPPTDSKEELVEKYFYASIYGMMKVLAKMGISTLASYKGAQIFEALGLSSEVIHKCFEGTPSRIEGATFEMLARDALRLHELAFPSRTPPPGSADAKALPNPGDYHWRKNGEVHLNDPLAMAKLQEAAKVNSREAYKEYSKRIQELNKACNLRGMLKFIDSTSKISLDEVEPASEIVKRFCTGAMSYGSISLEAHTALAVAMNKLGGKSNTGEGGEQPSRMEPLPDGSMNPKRSAIKQVASGRFGVSSYYLTNADGLQIKMAQGAKPGEGGELPGHKVIGDIAVTRHSTAGVGLISPPPHHDIYSIEDLAQLIHDLKNSNPQARISVKLVSEAGVGVVASGVVKGHADHVLISGHDGGTGASRWTGIKNAGLPWELGLAETHQTLVANGLRGRAVLQTDGQLKTGRDVAVACLLGAEEFGFSTAPLITLGCIMMRKCHTNTCPVGIATQDPVLREKFAGEPEHVINFFFMLAEELREIMAQLGLRTINEMVGRSDMLEVDPEVVKSNEKLENIDLSLILKPAAEIRPGAAQYCVEKQDHGLDMALDNKLIALSRAALEKEVRVFIETPIKNTNRAVGTTLSHEVTKRYHMKGLDPGTIHVKLTGSAGQSFGAFLCPGITLELEGDSNDYVGKGLSGGKIVVYPPRNSTFSAEDNIVIGNVALYGATKGEAYFNGMAAERFCVRNSGARTVVEGIGDHGCEYMTGGTVVILGKTGRNFAAGMSGGIAYVYDVDGTFSVRCNNELVDLYHVEEEDDVTTLKMMIEQHRLHTESVLAKDILSKFDTLLPKFVKVYPRDYKRVLEEMKAEKAAARPTKEPKVANGVSVTTKKIQTEKSSSRPTRVANAKKYRGFVTYEREGVSYRDPNERVKDWNEVAIESVPGPLLNTQSARCMDCGTPFCHQESSGAGCPLGNKIPEFNELVHQNRWREALDRLLETNNFPEFTGRVCPAPCEGSCVLGIIENPVSIKSIECSIIDKGFEEGWMVPRPPLQRTGKKIAIVGSGPAGLAAADQLNKMGHFVTVFERSDRIGGLMMYGVPNMKTDKIGVVQRRVNLMAEEGVTFVVNANVGSDPLYSIERLHSENNAVILACGATKPRDLSIPGRELAGVHFAMEFLHANTKSLLDSNLEDGRYISAQGKKVVVIGGGDTGTDCIGTSVRHGCSSIVNLELLTKPPSKRASDNPWPQWPRVFRVDYGHQEASTKFGNDPRTYEVLTKRFIGDEDGKLKALEVVRVKWEKVDGKFQFKEIEGSQEIIEADLVLLAMGFLGPEENIADKLGLEKDNRSNFKAQFGHFGTSVDGVFAAGDCRRGQSLVVWAITEGREAAAAVDKYLSRDEQNVAGLT

SEQ ID NO: 2 - DNA, 10173 bp, Triticum aestivum
atgccgacggcgcagggggattggtttgaagcacgcggcgccgccgggcggccgcagggcccgccgcagccagtctgcctcggcgccgtgccgctccgcaaggcaggcgcacggcgccatgtccctggatggcgggttcctcggcggcgcgcagcgcaccgaggaacgcgtcgcgccacgcccgcctcgggccgcggcgcgcgacgccgagtccatcaggcccatgtctctgctacccgagagcagcattgggctgtacgacccggcgttcgagcgtgactcgtgcggcgttggcttcgtcgccgagctgtcgggcgttgacaaccgggcgaccgtgagttctttgcaccaggacaccgccgcagcttctctcttttatctaccacgttgagatattgattttgctgtgatttactttacaggtcgtcgatgccattcagatgcttgaaagaatggcacaccgaggtgcctgcggctgtgagaaaaacactggtgatggtgccggcattctcgttgctctaccacacaccttcttccgagaggtgaataaacagaaaatttcaagcaaactcagttttccttgacagagttattcatcttgggtgttgctttgctattgctgcaggtgacaaaggatgccggtttcgagttaccgccaccaggtgagtatgctgttggaatggtcttcctgccaaccgatgagaagcgccgcgagaggagcaaaactgagtttacaaaggttggttggttggtgtatagacaattagacagcgtgcgaggcgagtctgcgatggtaagcttatgtcttggattgtttttattcatgcaggtcgctgagtctctaggacattcgatacttgggtggcgccaggttcccactgacaattcagacttgggccaagctgcgctcgatactgaaccagccattgaacaggttttcctcaccaagagttcaaaatcgaaggccgacttcgaacagcaggtcctccatgagtttaattttctctcaacacatgtaatgagctttcctctcaactaacccatcaattatcacagttgtttatcctgaggaggctctcaattgtatctatccgggccgcgctgaatcttcagcgtggaggagagagagatttctacatgtgctctctatcttcaaggtccttacttcttctgaattgcattttcactacaaactgatgaggttgagatcagggagcccaattaagctcctgactttaatggaacattaacaacacaaaagttgataggaaagctgactgggaaaaacaatcctgggctatctcttaagcattagaaatctgtaatgtaaggacatttcatttttgctgtattcatgtgagtaattattgttaaatcttctattgtggttttccaggaccattgtttacaagggccaacttatgccatcccagctccaagggtactactatgcggatataggtcatgaaaacttctccagttatatggctctggtaaatacgaaaccttgatactcttacaacaaaagtcatggtcgtagtcagcggatgggaatcaataatagcaagccagttgtaagatcaagactcgctcctgtgcagacatcaaatttgagctgtctattcatatgctatttcagtcctgcttcgtgatgcctttaagtgaagtactagaaacttctcttagttgatacccgataaccatgttgcagagtccagctgcagttctgctatggccgactgcttgccgactcttcaggatgtgaccgaagaaattgtgtttggtttgaggtagcttagggctcccaactgttttgcatgaaattatacgcagctgacgtatttttacaaaattcgttcttcacaaggaagttttgttttgaatatctgcaaaagatgtttataatatgatttctaaaagtcatatcttgaaacaggttcactcaaggttctccaccaacaccttccccagctgggaccgtgcacagccaatgcgtgtcttaggccacaatggagagatcaatactctcaaagggaacaaaaactggtaatattattgtcaaatcttttcctctttgctttaaccttttcttggcagatgcaactatctcaatatgcacgttgtatatatggcaggatgaaagctcgtgagggtctcttggagtgtgagaagcttggcctatcacaggatgaaatgtcaaagattcttccaatagtagatgccacttcttcagattcaggtccgcagtctaatatgcagcatgaattccacttggcaaataaaaattcatctcggttttgtgagttactaagtgtgaaaaaacatgtgaattctaggtgcatttgatggtgttctcgagctccttatccgtggtggaagaagcctgccagaagctgtgatgatgatgatccctgaggcgtggcagaatgatgtaaacatggaacctgataagaaagctctgtacgagttcttgtcagcccttatggagccttgggatggacctgctctcatatcttgtaaaaaacttaaacccgtttcttttttctgatcatacttctaactgattacttaagctaacatgggtacaacctgtagttaccgatggccgctaccttggagctaccctggatcgcaatggccttaggcctggtcgattttatgtaacccacagtggacgtgtggtcatgggtagtgaagttggtgttgtggatattcctgcccaagatgtgttgagaaagggtcggcttaaccctggaatgatgttactcgttgacttcgacaaccatactgtagtagatgatgaagcactcaaggcacagtactctaaagctcacccgtatggagaatggctcaagcgacaaaagatgtacctcaaagacattgtagaatctgtcccagaaactgacagagttgctccaagcatttctggttctattacggtaagtacttacgtggtccaacttcctcattcactgggtagtttggtgatgaaaattggtaaaccataaggaacatggtgattttaatattcttttacaatttggtgtagcaaacaaatgagaacaaggaatgtgtaggcatcaatgcaattgtgactccactaaaggcgtttgggtaagcaaggattgaacattgtcaactatcactcatgtttcaatttctgctggctaatgatccaaccgattaactacaggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgcgagggatacaaaattttagttctttcagacagaggtcagtgacttattcactttatacttgcatcccattgtttgatggtacaaattactcataagttgacaaaaaattctagcagatgggtcaggtgtgcacaatttattatcgttagcttccagcaaacatatcacatcaccatatagtaacttaaatatgatcataattttgtttgatagtcaattgtttgcagtgtaatgtgttgtatgaaggacattcttactttgtcaccttggacataattggaatgtatattaagtttattatgcaactcaacatcttccaatactgaaatttgttgtattttcttgattcaggattttcttcagaccgtgttgctgtcagttccctcttagcagttggagcagtacatcaacaccttgttgcaaatcttgagaggacacgcgtaggattattggttgagtctgctgaacctcgtgaagtgcaccatttctgtacactcgttggatttggtgcagatgctatatgcccttatttggccattgaagcaatttggtgcttgcagactgatgggaagattccccctaccgactcaaaggaggaacttgtcgaaaaatatttttatgcttccatctatggaatgatgaaggttcttgcaaagatgggaatatccacccttgcatcttacaaaggggcacagatttttgaagctcttggactttcttctgaagtgattcacaagtgttttgaaggcactcctagcagaattgagggtgcaacgttcgaaatgcttgcacgtgatgctctccgtcttcacgagttggcattcccatcaagaacacctccgcctggcagtgcagatgcaaaagcccttcccaatccaggggattaccactggaggaaaaatggtgaagtccatctaaatgatcctcttgcaatggcaaaattacaagaagcagctaaagtaaacagccgggaagcatacaaagaatattctaagcgaattcaagagcttaacaaggcgtgcaatctgcgtggtatgctgaaatttatagatagcactagcaagatttctttggatgaggttgaacctgcaagtgagatagtgaaacgcttttgtactggggcaatgagttatggatctatttcgttggaggcacatactgcccttgctgtggccatgaacaaactgggaggcaaatctaacacaggtatttcatgcatatgtgacattttttaccctttggctggttgttacccttctgatgaaatcatgtatagtagctttacttgcacatcaactcttagagccttgttgcataacatatttttcttatttttgctgcagtagtgtagtgtctatgtttctactgtttaaatatgccctcgaagttggtactttatacatgaccatgtctagaaatgcactacttattttgatgctatcacatgaaaactatactctcatatttacttgtcgacatacttgtactgcacttgtgtgtttggttgcattctcgtctcagcatagttccctcttcatgcatgctcaactcaacacccctcttcatgcatgctttcttcatgcatgcttctagtgtatttgtgctaagctagtgtggactcatgcaccctccatacactctaccaaacacccaaaagtgggtcgagaagggaactcttgggctcatgcacccttcatacaccctaccaaacacacccttaatgcgctaagcttttcatcttcctgttgatgttgaatgcacttctgtgaacaaattatcccagttcatagcaagagttaacttctaagcttttatctatctgttgatgttgaatgcacttctggtagcaaattatcctaattcctagcaagatttaacttctgcttgcgtagaaacccagacagcttgcacgcactattcttctttggactgtttgtattagatattttctgcccacacagttaatattatattgccaaatctttgtatttattttcgctcctaaactaaactcattttacccatctgctaatttaggggagggaggggagcagccttctcgtatggagcctcttcctgatggttcgatgaatccaaaacgaagtgcaatcaagcaagttgccagtggacgatttggagtttccagctattatctgactaatgcagatgggctgcagataaaaatggctcaggtacttgacaatgctgtgcattgcgcaatcactggaatgatctcagtttatagaaagcaactgtattcaacgtcctgtggttggcatttattctttttttgtatcgtatatgtatagggtgctaagcctggtgaaggtggtgagcttccaggtcacaaggttatcggtgacattgcagttaccagacattctacagctggtgttgggcttattagtccacctcctcaccatgatatatattctatcgaggatcttgcacagcttatccatgaccttaaggtacattacctgatgctcttctatagccaaggcatctgcctatttatgtatggttgttagttgctacttctagattatattggttatctaaacgctgtgatggtctaaccctcctgagaagctagaatcacttgctaaactggtacaagtgatcctgcacatatgcatgtgtctccatttgggggtttaccctcctttttgaaaaactcattttgcaaatgttacctgctgatgtagtgatattcatgcctgttttctctctgcagaattcaaatcctcaagctcgaatcagcgtgaagctagtgtctgaggctggtgttggagttgtagctagcggtgttgtaaaaggacacgcagaccatgttcttatttctggccatgacggtggcaccggtgcatcaagatggacaggtatcaagaatgctggacttccatgggaactaggattggctgagacacatcaaactttagtggcaaatgggcttcgtggtcgagctgtcctacaaacagatggccaattaaaaactggtagagatgttgctgtggcgtgcttacttggtgcagaggaatttggtttcagcactgctccactgatcacacttggctgcattatgatgcggaagtgccataccaatacttgccctgttggtatagccactcaagatccagtgctccgagaaaaatttgctggagaaccagagcatgtcattaactttttcttcatgcttgctgaggagctaagagaaattatggctcagcttggcctccgtacaattaatgaaatggttggacgctctgatatgcttgaagttgatccagaagtagttaagagcaatgagaaacttgaaaatattgatctctcattgatcttaaaaccagctgcagagattcgtcctggggctgctcagtactgtgttgaaaagcaagaccatggccttgacatggctttggataacaaacttatagctttatcaagggctgcacttgaaaaggaagttcgtgttttcattgagactccaatcaagaacactaatcgggcagtaggcaccacacttagccatgaagtcacaaaacgttatcacatgaagggattagatcctggaaccattcatgtgaaactcactggaagtgctggtcagagttttggtgcttttctctgtcctggaataacccttgagcttgaaggagacagcaatgattacgttggcaaaggattatctggtggaaagattgttgtgtacccacccaggaacagtacatttagtgcagaagacaatattgtcattggtaatgtggccctgtatggtgctaccaagggagaagcatacttcaatggaatggcagcagaaaggttttgtgttcgtaattctggtgctcgaacagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagaggatgatgtaaccactttgaaaatgatgatagagcaacatcgacttcacacagagagtgtcctggccaaagacatactctctaaattcgacactcttcttccaaaatttgtaaaagtatacccaagggattataagagggttctagaagaaatgaaggcagaaaaagctgcagctaggcctacaaaggaacctaaggtggcaaatggtgtttctgtgacaactaaggtaatatatgatttaagtaataattcttctgcatacctttttaatttgaatttatagtagagatggtgttctccatttaggtttatccatcagaaattgcaatgatacacattgccactactgtgtgaacttctatttttgcctaagtagattaactttgatctacagaaaatacaaacagagaagtcatccagccgaccaacacgtgttgccaacgccaaaaagtaccggggttttgtaacatatgagcgagagggtgtttcttaccgtgatccaaatgagcgtgttaaagattggaacgaggttgccattgaatcagttccagggccactgttaaacacacagtctgctcgttgtatggattgtggcactcctttctgtcatcaggtgaagctgagttcatcttttctatttaacattgatgattgtagttggatttattgtggggttaaggattgatatgtttagtcattgactacatatgacattgagatgctaagatttcatgattatttgtacaggaaagctcaggtgctggctgtcctcttggaaataagatcccagagttcaatgaattagttcaccagaatagatggcgtgaagcattggatcgcctactagagacaaacaacttccctgaatttactggacgtgtgtgccctgctccttgtgaggggtcttgtgttcttggcattattgagaacccagtatcaatcaaaagcatagaatgctcgattatagacaaaggttttgaagagggatggatggtaccacgaccaccacttcaaagaacagggtatgtcttttttaccatctagttcggcttattcccttatttatttacttctgtttcattaataggtaacatttgagttacactttctaatataaactgttcctttcgcagaaagaaaatcgctatagttggcagtggtcctgctggtttggctgccgctgatcaactaaataaaatgggccattttgtaactgtatttgaacgttcggatcgtataggaggtcttatgatgtatggagtaccaaacatgaagacagacaagattggagttgttcagcgtcgtgtcaatttaatggccgaagagggtgtaacatttgtggtgaatgctaatgtagggagtgatcctttatactcaattgaacgtctccattctgagaacaatgcagttattttggcttgtggagctacaaaaccaaggtaactaattggaacgaggattttttttttctagtttactccaagagtagcaacaatctcaaaagaactgacatatatcttttgacactaactgactaacatcatgtgggatattcttgaacagggacctcagtattcctggccgtgagctagctggagttcattttgccatggaatttctccacgcaaataccaaaagtttgcttgatagcaacctggaggatggaagatacatatctgcccagggtaagaaggtggtggtcattggtggtggagacacaggcacagattgcatcggtacgtctgttaggcatggttgcagcagcattgtaaatctggagcttctcaccaagccaccaagcaagagagcttctgacaacccctggccccaggtaaatgcaaaaaaaaaaaaactggatatttccctgcacatgtgaatttggccattccatttccagcttggaaagttcttaagtcatcattctgtttttgcatgctgtggcctagagtcttccgagtggactatgggcaccaggaagcatctaccaaatttggaaatgatccaagaacttactaagtcttaaccaagcgtttcattgggtgatgaagatggaaaattgaaggcccttgaggtggtgcgcgtgaagtgggagaaagtagatggaaaattccagttcaaggagattgaaggatcacaagagatcatcgaggcagaccttgtcctccttgccatgggattcctgggccctgaagaggttagttatgcagactctgaactactgtcacgcctgagttttatcttgatgataaatgttggacaaacaactcaccgttttatttctctgcagaacatcgctgacaaactgggtttagagaaagacaaccgttccaacttcaaagctcaattcggacacttcgggaccagtgtggatggcgtttttgccgctggggattgcaggcgcgggcaatcgctggttgtttgggccatcaccgaagggcgtgaagctgctgctgcagtagataagtacttgtcaagggatgaacaaaatgtcgcaggccttaca

SEQ ID NO: 3 - DNA, 8964 bp, Triticum aestivum
  misc_feature (913)..(913): n is a, c, g, or t
gaggaggctctcaattgtatctatccgggccgcgctgaatcttcagcgtggaggagagagagatttctacatgtgctctctatcttcaaggtccttacttcttctgaattgcattttcactacaaactgatgaggttgagatcagggagcccaattaagctcctgactttaatggaacattaacaacacaaaagttgataggaaagctgactgggaaaaacaatcctgggctatctcttaagcattagaaatctgtaatgtaaggacatttcatttttgctgtattcatgtgagtaattattgttaaatcttctattgtggttttccaggaccattgtttacaagggccaacttatgccatcccagctccaagggtactactatgcggatataggtcatgaaaacttctccagttatatggctctggtaaatacgaaaccttgatactcttacaacaaaagtcatggtcgtagtcagcggatgggaatcaataatagcaagccagttgtaagatcaagactcgctcctgtgcagacatcaaatttgagctgtctattcatatgctatttcagtcctgcttcgtgatgcctttaagtgaagtactagaaacttctcttagttgatacccgataaccatgttgcagagtccagctgcagttctgctatggccgactgcttgccgactcttcaggatgtgaccgaagaaattgtgtttggtttgaggtagcttagggctcccaactgttttgcatgaaattatacgcagctgacgtatttttacaaaattcgttcttcacaaggaagttttgttttgaatatctgcaaaagatgtttataatatgatttctaaaagtcatatcttgaaacaggttcactcaaggttctccaccaacaccttccccagctgggaccntgcacagccaatgcgtgtcttaggccacaatggagagatcaatacnctcaaagggaacaaaaactggtaatattattgtcaaatcttttcctctttgctttaaccttttcttggcagatgcaactatctcaatatgcacgttgtatatatggcaggatgaaagctcgtgagggtctcttggagtgtgagaagcttggcctatcacaggatgaaatgtcaaagattcttccaatagtngatgccacttcttcagattcaggtccgcagtctaatatgcagcatgaattccacttggcaaataaaaattcatctcggttttgtgagttactaagtgtgaaaaaacatgtgaattctaggtgcatttgatggtgttctcgagctccttatccgtggtggaagaagcctgccagaagctgtgatgatgatgatccctgaggcgtggcagaatgatgtaaacatggaacctgataagaaagctctgtacgagttcttgtcagcccttatggagccttgggatggacctgctctcatatcttgtaaaaaacttaaacccgtttcttttttctgatcatacttctaactgattacttaagctaacatgggtacaacctgtagttaccgatggccgctaccttggagctaccctggatcgcaatggccttaggcctggtcgattttatgtgacccacagtggacgtgtggtcatgggtagtgaagttggtgttgtggatattcctgcccaagatgtgttgagaaagggtcggcttaaccctggaatgatgttactcgttgacttcgacaaccatactgtagtagatgatgaagcactcaaggcacagtactctaaagctcacccgtatggagaatggctcaaacgacaaaagatgtacctcaaagacattgtagaatctgtcccagaaactgacagagttgctccaagcatttctggttctattacggtaagtacttacgtggtccaacttcctcattcactgggtagtttggtgacgaaaattggtaaaccataagtaacatggtgattttaatattcttttacaatttggtgtagcaaacaaatgagaacaaggaatgtgtaggcatcaatgcaattgtgactccactaaaggcgtttgggtaagcaaggattaaacattgtcaactatcactcatgtttcagtttctgctggctaatgatccaaccgattaactacaggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgngagggatacaaaattttagttctttcagacagaggtcagtgacttattcactttatacttgcatcccattgtttgatggtacaaattactcataagttgacaaaaaattctagcagatgggtcaggtgtgcacaatttattatcgttagcttccagcaaacatatcacatcaccatatagtaacttaaatatgatcataattttgtttgatagtcaattgtttgcagtgtaatgtgttgtatgaaggacattcttactttgtcaccttggacataattggaatgtatattaagtttattatgcaactcaacatcttccaatactgaaatttgttgtattttcttgattcaggattttcttcagaccgtgttgctgtcagttccctcttagcagttggagcagtacatcaacaccttgttgcaaatcttgagaggacacgcgtaggattattggttgagtctgctgaacctcgtgaagtgcaccatttctgtacactcgttggatttggtgcagatgctatatgcccttatttggccattgaagcaatttggtgcttgcagactgatgggaagattccccctaccgactcaaaggaggaacttgtcgaaaaatatttttatgcttccatctatggaatgatgaaggttcttgcaaagatgggaatatccacccttgcatcttacaaaggggcacagatttttgaagctcttggactttcttctgaagtgattcacaagtgttttgaaggcactcctagcagaattgagggtgcaacgttcgaaatgcttgcacgtgatgctctccgtcttcacgagttggcattcccatcaagaacacctccgcctggcagtgcagatgcaaaagcccttcccaatccaggggattaccactggaggaaaaatggtgaagtccanctaaatgatcctcttgcaatggcaaaattacaagaagcagctaaagtaaacagccgggaagcatacaaagaatattctaagcgaattcaagagcttaacaaggcgtgcaatctgcgtggtatgctgaaatttatagatagcactagcaagatttctttggatgaggttgaacctgcaagtgagatagtgaaacgcttttgtactggggcaatgagttatggatctatttcgttggaggcacatactgcccttgctgtggccatgaacaaactgggaggcaaatctaacacaggtatttcatgcatatgtgacattttttaccctttcgctggttgttacccttctgatgaaatcatgtatagtagctttacttgcacatcaactcttagagccttgttgcataacatatttttcttatttttgctgcagtagtgtagtgtctatgtttctactgtttaaatatgccctcgaagttggtactttatacatgaccatgtctagaaatgcactacttattttgatgctatcacatgaaaactatactctcatagcagtacaagtatttatcttcctgttgatgttgaatgcacttctgtgaaaactatactctcatagtagtgtctatgtttctacaagtatttatcacatgaaaactatactcacataaaaccatttcatcttcctgttgatgttgaatgcacttctgtgaacaaattatcccagttcatagcaagagttaacttctaagcttttatctatctgttgatgttgaatgcacttctggtagcaaattatcctaattcctagcaagatttaacttctgcttgcgtagaaacccagacagcttgcacgcattattcttctttggactgtttgtattatagatattttctgcccacacagttaatattatattgccaaatctttgtatttattttcactcctaaactaaactcattttacccatctgctaatttaggggagggaggggagcagccttctcgtatggagcctcttcctgatggttcgatgaatccaaaacgaagtgcaatcaagcaagttgccagtggacgatttggagtttccagctattatctgactaatgcagatgagctgcagataaaaatggctcaggtacttgacaatgctgtgcattgcgcaatcactggaatgatctcagtttatagaaagcaactgtattcaacgtcctgtggttggcatttattctttttttgtatcgtatatgtatagggtgctaagcctggtgaaggtggtgagcttccaggtcacaaggttatcggtgacattgcagttaccagacattctacagctggtgttgggcttattagtccacctcctcaccatgatatatattctatcgaggatcttgcacagcttatccatgaccttaaggtacattacctgatgctcttctatagccaaggcatctgcctatttatgtatggttgttagttgctacttctagattatattggttatctaaacgctgtgatggtctaaccctcctgagaagctagaatcacttgctaaactggtacaagtgatcctgcacatatgcatgtgtctccatttgggggtttaccctcctttttgaaaaactcattttgcaaatgttacctgctgatgtagtgatattcatgcctgttttctctctgcagaattcaaatcctcaagctcgaatcagcgtgaagctagtgtctgaggctggtgttggagttgtagctagcggtgttgtaaaaggacacgcagaccatgttcttatttctggccatgacggtggcaccggtgcatcaagatggacaggtatcaagaatgctggacttccatggggaactaggattggctgagacacatcaaactttagtggcaaatgggcttcgtggtcgagctgtcctacaaacagatggccaattaaaaactggtagagatgttgctgtggcgtgcttacttggtgcagaggaatttggtttcagcactgctccactgatcacacttggctgcattatgatgcggaagtgccataccaatacttgccctgttggtatagccactcaagatccagtgctccgagaaaaatttgctggagaaccagagcatgtcattaactttttcttcatgcttgctgaggagctaagagaaattatggctcagcttggcctccgtacaattaatgaaatggttggacgctctgatatgcttgaagttgatccagaagtagttaagagcaatgagaaacttgaaaatattgatctctcattgatcttaaaaccagctgcagagattcgtcctggggctgctcagtactgtgttgaaaagcaagaccatggccttgacatggctttggataacaaacttatagctttatcaaggnctgcacttgaaaaggaagttcgtgttttcnttgagactccaatcaagaacactaatcgggcagtaggcacnacacttagccatgaagtcacaaaacgttatcacatgaagggattagatcctggaaccattcatgtgaaactcactggaagtgctggtcagagttttggtgcttttctctgtcctggaataacccttgagcttgaaggagacagcaatgattacgttggcaaaggattatctggtggaaagattgttgtgtacccacccaggaacagtacatttagtgcagaagacaatattgtcattggtaatgtggccctgtatggtgctaccaagggagaagcatacttcaatggaatggcagcagaaaggttttgtgttcgtaattctggtgctcggacagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagaggatgatgtaaccactttgaaaatgatgatagagcaacatcgacttcacacagagagtgtcctggccaaagacatactctctaaattcgacactcttcttccaaaatttgtaaaagtatacccaagggattataagagggttctagaagaaatgaaggcagaaaaagctgcagctaggcctacaaaggaacctaaggtggcaaatggtgtttctgtgacaactaaggtaatatatgatttaagtaataattcttctgcatacctttttaatttgaatttatagtagagatggtgttctccatttaggtttatccatcagaaattgcaatgatacacattgccactactgtgtgaacttctatttttgcctaagtagattaactttgatctacagaaaatacaaacagagaagtcatccagccgaccaacacgtgttgccaacgccaaaaagtaccggggttttgtaacatatgagcgagagggtgtttcttaccgtgatccaaatgagcgtgttaaagattggaacgaggttgccattgaatcagttccagggccactgttaaacacacagtctgctcgttgtatggattgtggcactcctttctgtcatcaggtgaagctgagttcatcttttctatttaacattgatgattgtagttggatttattgtggggttaaggattgatatgtttagtcattggctacatatgacattgagatgctaagatttcatgattatttgtacaggaaagctcaggtgctggctgtcctcttggaaataagatcccagagttcaatgaattagttcaccagaatagatggcgtgaagcattggatcgcctactagagacaaacaacttccctgaatttactggacgtgtgtgccctgctccttgtgaggggtcttgtgttcttggcattattgagaacccagtatcaatcaaaagcatagaatgctcgattatagacaaaggttttgaagagggatggatggtaccacgaccaccacttcaaagaacagggtatgtcttttttaccatctagttcggcttattcccttatttatttacttctgtttcattaataggtaacatttgagttacactttctaatataaactgttcctttcgcagaaagaaaatcgctatagttggcagtggtcctgctggtttggctgccgctgatcaactaaataaaatgggccattttgtaactgtatttgaacgttcggatcgtataggaggtcttatgatgtatggagtaccaaacatgaagacagacaagattggagttgttcagcgtcgtgtcaatttaatggccgaagagggtataacatttgtggtgaatgctaatgtagggagtgatcctttatactcaattgaacgtctccgttctgagaacaatgcagttattttggcttgtggagctacaaaaccaaggtaactaattggaacgaggattttttttttctagtttactccaagagtagcaacaatctcaaaagaactgacatatatcttttgacactaactgactaacatcatgtgggatattcttgaacagggacctcagtattcctggccgtgagctagctggagttcattttgccatggaatttctccacgcaaataccaaaagtttgcttgatagcaacctggaggatggaagatacatatctgcccagggtaagaaggtggtggtcattggtggtggagacacaggcacagattgcatcggtacgtctgttaggcatggttgcagcagcattgtaaatctggagcttctcaccaagccaccaagcaagagagcttctgacaacccctggccccaggtaaatgcaaaaaaaaaaaactggatatttccctgcacatgtgaatttggccattccatttccagcttggaaagttcttaagtcatcattctgtttttgcatgcagtggcctagagtcttccgagtggactatgggcaccaggaagcatctaccaagtttggaaatgatccaagaacttacgaagtcttaaccaagcgtttcattggtgatgaagatggaaaattgaaggcccttgaggtggtgcgcgtgaagtgggagaaagtagatggaaaattccagttcaaggagattgaaggatcacaagagatcatcgaggcagaccttgtcctccttgccatgggattcctgggccctgaagaggttagttatgcagactctgaactactgtcacgcctgagttttatcttgatgataaatgttggacaaacaactcaccgttttatttctctgcagaacatcgctgacaaactgggtttagagaaagacaaccgttccaacttcaaagctcaattcggacacttcgggaccagtgtggatggcgtttttgccgctggggattgcaggcgcgggcaatcgctggttgtttgggccatcaccgaagggcgtgaagctgctgctgcagtagataagtacttgtcaagggatgaacaaaatgtcgcaggccttacatag

SEQ ID NO: 4 - DNA, 9113 bp, Triticum aestivum
  misc_feature (1140)..(1140): n is a, c, g, or t
atcctgaggaggctctcaattgtatctatccgggccgcgctgaatcttcagcgtggaggagagagagatttctacatgtgctctctatcttcaaggtccttacttcttctgaattgcattttcactacaaactgatgaggttgagatcagggagcccaattaagctcctgactttaatggaacattaacaacacaaaagttgataggaaagctgactgggaaaaacaatcctgggctatctcttaagcattagaaatctgtaatgtaaggacatttcatttttgctgtattcatgtgagtaattattgttaaatcttctattgtggttttccaggaccattgtttacaagggccaacttatgccatcccagctccaagggtactactatgcggatataggtcatgaaaacttctccagttatatggctctggtaaatacgaaaccttgatactcttacaacaaaagtcatggtcgtagtcagcggatgggaatcaataatagcaagccagttgtaagatcaagactcgctcctgtgcagacatcaaatttgagctgtctattcatatgctatttcagtcctgcttcgtgatgcctttaagtgaagtactagaaacttctcttagttgatacccgataaccatgttgcagagtccagctgcagttctgctatggccgactgcttgccgactcttcaggatgtgaccgaagaaattgtgtttggtttgaggtagcttagggctcccaactgttttgcatgaaattatacgcagctgacgtatttttacaaaattcgttcttcacaaggaagttttgttttgaatatctgcaaaagatgtttataatatgatttctaaaagtcatatcttgaaacaggttcactcaaggttctccaccaacaccttccccagctgggaccgtgcacagccaatgcgtgtcttaggccacaatggagagatcaatactctcaaagggaacaaaaactggtaatattattgtcaaatcttttcctctttgctttaaccttttcttggcagatgcaactatctcaatatgcacgttgtatatatggcaggatgaaagctcgtgagggtctcttggagtgtgagaagcttggcctatcacaggatgaaatgtcaaanattcttccaatagtngatgccacttcttcagattcaggtccgcagtctaatatgcagcatgaattccacttggcaaatanaaattcatctcggttttgngagttactaagtgtgaaaaaacatgtgaattctaggtgcatttgatggtgttctngagctccttatccgtggtggaagaagcctgccagaagctgtgatgatgatgatcccngaggcgtggcagaatgatgtaaacatggaacctgataagaaagctctgtacgagttcttgtcagcccttatggagccttgggatggacctgctctcatatcttgtaaaaaacttaaacccgtttcttttttctgatcatacttctaactgattacttaagctaacatgggtacaacctgtagttaccgatggccgctaccttggagctaccctggatcgcaatggccttaggcctggtcgattttatgtaacccacagtggacgtgtggtcatgggtagtgaagttggtgttgtggatattcctgcccaagatgtgttgagaaagggtcggcttaaccctggaatgatgttactcgttgacttcgacgaccatactgtagtagatgatgaagcactcaaggcacagtactctaaagctcacccgtatggagaatggctcaagcgacaaaagatgtacctcaaagacattgtagaatctgtcccagaaactgacagagttgctccaagcatttctggttctattacggtaagtacttacgtggtccaacttcctcattcactgggtagtttggtgatgaaaattggtaaaccataaggaacatggtgattttaatattcttttacaatttggtgtagcaaacaaatgagaacaaggaatgtgtaggcatcaatgcaattgtgactccactaaaggcgtttgggtaagcaaggattgaacattgtcaactatcactcatgtttcaatttctgctggctaatgatccaaccgattaactacaggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgcaagggatacaaaattttagttctttcagacagaggtcagtgacttattcactttatacttgcatcccattgtttgatggtncaaattactcataagttgacaaaaaattctagcagatgggtcaggtgtgcacaatttattatcgttagcttccagcaaacatatcacatcaccatatantaacttaaatatgatcataattttntttgatagtcaattgtttncagtgtaatgtgttgtatgaaggacattcttactttgtcaccttggacataattggaatgtatattaagtttattatgcaactcaacatcttccaatactgaaatttgttgtattttcttgattcaggattttcttcagaccgtgttgctgtcagttccctcttagcagttggagcagtacatcaacaccttgttgcaaatcttgagaggacacgcgtaggattattggttgagtctgctgaacctcgtgaagtgcaccatttctgtacactcgttggatttggtgcagatgctatatgcccttatttggccattgaagcaatttggtgcttgcagactgatgggaagattccccctaccgactcaaaggaggaacttgtcgaaaaatatttttatgcttccatctatggaatgatgaaggttcttgcaaagatgggaatatccacccttgcatcttacaaaggggcacagatttttgaagctcttggactttcttctgaagtgattcacaagtgttttgaaggcactcctagcagaattgagggtgcaacgttcgaaatgcttgcacgtgatgctctccgtcttcacgagttggcattcccatcaagaacacctccgcctggcagtgcagatgcaaaagcccttcccaatccaggggattaccactggaggaaaaatggtgaagtccatctaaatgatcctcttgcaatggcaaaattacaagaagcagctaaagtaaacagccgggaagcatacaaagaatattctaagcgaattcaagagcttaacaaggcgtgcaatctgcgtggtatgctgaaatttatagatagcactagcaagatttctttggatgaggttgaacctgcaagtgagatagtgaaacgcttttgtactggggcaatgagttatggatctatttcgttggaggcacatactgcccttgctgtggccatgaacaaactgggaggcaaatctaacacaggtatttcatgcatatgtgacattttttaccctttggctggttgttacccttctgatgaaatcatgtatagtagctttacttgcacatcaactcttagagccttgttgcataacatatttttcttatttttgctgcagtagtgtagtgtctatgtttctactgtttaaatatgccctcgaagttggtactttatacatgaccatgtctagaaatgcactacttattttgatgctatcacatgaaaactatactctcatatttacttgtggacatacttgtactgcacttgtgtgtttggttgcattctcgtctcagcatagttccctcttcatgcatgctcaactcaacacccctcttcatgcatgctttcttcatgcatgcttctagtgtatttgtgctaagctagtgtggactcatgcaccctccatacactctaccaaacacccaaaagtgggtcgagaagggaactcttgggctcatgcacccttcatacaccctaccaaacacacccttaatgcgctaagcttttcatcttcctgttgatgttgaatgcacttctgtgaacaaattatcccagttcatagcaagagttaacttctaagcttttatctatctgttgatgttgaatgcacttctggtagcaaattatcctnattcntagcaagatttaacttctgcttgcgtagaaacccagacagcttgcacgcactattcttctttggactgtttgtattagatattttctgcccacacagttaatattatattgccaaatctttgtatttattttcgctcctaaactaaactcattttacccatctgctaatttaggggagggaggggagcagccttctcgtatggagcctcttcctgatggttcgatgaatccaaaacgaagtgcaatcaagcaagttgccagtggacgatttggagtttccagctattatctgactaatgcagatgagctgcagataaaaatggctcaggtacttgacaatgctgtgcattgcgcaatcactggaatgatctcagtttatagaaagcaactgtattcaacgtcctgtggttggcatttattctttttttgtatcgtatatgtatagggtgctaagcctggtgaaggtggtgagcttccaggtcacaaggttatcggtgacattgcagttaccagacattctacagctggtgttgggcttattagtccacctcctcaccatgatatatattctatcgaggatcttgcacagcttatccatgaccttaaggtacattacctgatgctcttctatagccaaggcatctgcctatttatgtatggttgttagttgctacttctagattatattggttatctaaacgctgtgatggtctaaccctcctgagaagctagaatcacttgctaaactggtacaagtgatcctgcacatatgcatgtgtctccatttgggggtttaccctcctttttgaaaaactcattttgcaaatgttacctgctgatgtagtgatattcatgcctgttttctctctgcagaattcaaatcctcaagctcgaatcagcgtgaagctagtgtctgaggctggtgttggagttgtagctagcggtgttgtaaaaggacacgcagaccatgttcttatttctggccatgacggtggcaccggtgcatcaagatggacaggtatcaagaatgctggacttccatggggaactaggattggctgagacacatcaaactttagtggcaaatgggcttcgtggtcgagctgtcctacaaacagatggccaattaaaaactggtagagatgttgctgtggcgtgcttacttggtgcagaggaatttggtttcagcactgctccactgatcacacttggctgcattatgatgcggaagtgccataccaatacttgccctgttggtatagccactcaagatccagtgctccgagaaaaatttgctggagaaccagagcatgtcattaactttttcttcatgcttgctgaggagctaagagaaattatggctcagcttggcctccgtacaattaatgaaatggttggacgctctgatatgcttgaagttgatccagaagtagttaagagcaatgagaaacttgaaaatattgatctctcattgatcttaaaaccagctgcagagattcgtcctggggctgctcagtactgtgttgaaaagcaagaccatgccttgacatggctttggataacaaacttatagctttatcaaggnctgcacttgaaaaggaagttcgtgttttcnttgagactccaatcaagaacactaatcgggcagtaggcacnacacttagccatgaagtcacaaaacgttatcacatgaagggattagatcctggaaccattcatgtgaaactcactggaagtgctggtcagagttttggtgcttttctctgtcctggaataacccttgagcttgaaggagacagcaatgattacgttggcaaaggattatctggtggaaagattgttgtgtacccacccaggaacagtacatttagtgcagaagacaatattgtcattggtaatgtggccctgtatggtgctaccaagggagaagcatacttcaatggaatggcagcagaaaggttttgtgttcgtaattctggtgctcgaacagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagaggatgatgtaaccactttgaaaatgatgatagagcaacatcgacttcacacagagagtgtcctggccaaagacatactctctaaattcgacactcttcttccaaaatttgtaaaagtatacccaagggattataagagggttctagaagaaatgaaggcagaaaaagctgcagctaggcctacaaaggaacctaaggtggcaaatggtgtttctgtgacaactaaggtaatatatgatttaagtaataattcttctgcatacctttttaatttgaatttatagtagagatggtgttctccatttaggtttatccatcagaaattgcaatgatacacattgccactactgtgtgaacttctatttttgcctaagtagattaactttgatctacagaaaatacaaacagagaagtcatccagccgaccaacacgtgttgccaacgccaaaaagtaccggggttttgtaacatatgagcgagagggtgtttcttaccgtgatccaaatgagcgtgttaaagattggaacgaggttgccattgaatcagttccagggccactgttaaacacacagtctgctcgttgtatggattgtggcactcctttctgtcatcaggtgaagctgagttcatcttttctatttaacattgatgattgtagttggatttattgtggggttaaggattgatatgtttagtcattggctacatatgacattgagatgctaagatttcatgattatttgtacaggaaagctcaggtgctggctgtcctcttggaaataagatcccagagttcaatgaattagttcaccagaatagatggcgtgaagcattggatcgcctactagagacaaacaacttccctgaatttactggacgtgtgtgccctgctccttgtgaggggtcttgtgttcttggcattattgagaacccagtatcaatcaaaagcatagaatgctcgattatagacaaaggttttgaagagggatggatggtaccacgaccaccacttcaaagaacagggtatgtcttttttaccatctagttcggcttattcccttatttatttacttctgtttcattaataggtaacatttgagttacactttctaatataaactgttcctttcgcagaaagaaaatcgctatagttggcagtggtcctgctggtttggctgccgctgatcaactaaataaaatgggccattttgtaactgtatttgaacgttcggatcgtataggaggtcttatgatgtatggagtaccaaacatgaagacagacaagattggagttgttcagcgtcgtgtcaatttaatggccgaagagggtntaacatttgtggtgaatgctaatntagggagtgatcctttatactcaattgaacgtctccgttctgagaacaatgcagttattttggcttgtggagctacaaaaccaaggtaactaattggaacgaggattttttttttctagtttactccaagagtagcaacaatctcaaaagaactgncatatatcttttgacactaactgactaacatcatgtgggatattcttgaacagggacctcagtattcctggncgtgagctagctggagttcattttgccatggaatttctccacgcaaataccaaaagtttgcttgatagcaacctggaggatggaagatacatatctgcccagggtaagaaggtggtggtcattggtggtggagacacaggcacagattgcatcggtacgtctgttaggcatggttgcagcagcattgtaaatctggagcttctcaccaagccaccaagcaagagagcttctgacaacccctggccccaggtaaatgcaaaaaaaaaaaaactggatatttccctgcacatgtgaatttggccattccatttccagcttggaaagttcttaagtcatcattctgtttttgcatgcagtggcctagagtcttccgagtggactatgggcaccaggaagcatctaccaagtttggaaatgatccaagaacttacgaagtcttaaccaagcgtttcattggtgatgaagatggaaaattgaaggcccttgaggtggtgcgcgtgaagtgggagaaagtagatggaaaattccagttcaaggagattgaaggatcacaagagatcatcgaggcagaccttgtcctccttgccatgggattcctgggccctgaagaggttagttatgcagactctgaactactgtcacgcctgagttttatcttgatgataaatgttggacaaacaactcaccgttttatttctctgcagaacatcgctgacaaactgggtttagagaaagacaaccgttccaacttcaaagctcaattcggacacttcgggaccagtgtggatggcgtttttgccgctggggattgcaggcgcgggcaatcgctggttgtttgggccatcaccgaagggcgtgaagctgctgctgcagtagataagtacttgtcaagggatgaacaaaatgtcgcaggccttacatag

SEQ ID NO: 5 - DNA, 6432 bp, Triticum aestivum
atgccgacggcgcagggggattggtttgaagccgcggcgccgccgggcggccgcagggcccgccgcagccagtctgcctcggcgccgtgccgctccgcaaggcaggcgcacggcgccatgtccctggatggcgggttcctcggcggcgcgcagcgcaccgaggaacgcgtcgcgccacgcccgcctcgggccgcggcgcgcgacgccgagtccatcaggcccatgtctctgctacccgagagcagcattgggctgtacgacccggcgttcgagcgtgactcgtgcggcgttggcttcgtcgccgagctgtcgggcgttgacaaccgggcgaccgtcgtcgatgccattcagatgcttgaaagaatggcacaccgaggtgcctgcggctgtgagaaaaacactggtgatggtgccggcattctcgttgctctaccacacaccttcttccgagaggtgacaaaggatgccggtttcgagttaccgccaccaggtgagtatgctgttggaatggtcttcctgccaaccgatgagaagcgccgcgagaggagcaaaactgagtttacaaaggtcgctgagtctctaggacattcgatacttgggtggcgccaggttcccactgacaattcagacttgggccaagctgcgctcgatactgaaccagccattgaacaggttttcctcaccaagagttcaaaatcgaaggccgacttcgaacagcaggtgtttatcctgaggaggctctcaattgtatctatccgggccgcgctgaatcttcagcgtggaggagagagagatttctacatgtgctctctatcttcaaggaccattgtttacaagggccaacttatgccatcccagctccaagggtactactatgcggatataggtcatgaaaacttctccagttatatggctctggttcactcaaggttctccaccaacaccttccccagctgggaccgtgcacagccaatgcgtgtcttaggccacaatggagagatcaatactctcaaagggaacaaaaactggatgaaagctcgtgagggtctcttggagtgtgagaagcttggcctatcacaggatgaaatgtcaaagattcttccaatagtagatgccacttcttcagattcaggtgcatttgatggtgttctcgagctccttatccgtggtggaagaagcctgccagaagctgtgatgatgatgatccctgaggcgtggcagaatgatgtaaacatggaacctgataagaaagctctgtacgagttcttgtcagcccttatggagccttgggatggacctgctctcatatcttttaccgatggccgctaccttggagctaccctggatcgcaatggccttaggcctggtcgattttatgtaacccacagtggacgtgtggtcatgggtagtgaagttggtgttgtggatattcctgcccaagatgtgttgagaaagggtcggcttaaccctggaatgatgttactcgttgacttcgacaaccatactgtagtagatgatgaagcactcaaggcacagtactctaaagctcacccgtatggagaatggctcaagcgacaaaagatgtacctcaaagacattgtagaatctgtcccagaaactgacagagttgctccaagcatttctggttctattacgcaaacaaatgagaacaaggaatgtgtaggcatcaatgcaattgtgactccactaaaggcgtttgggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgcgagggatacaaaattttagttctttcagacagaggattttcttcagaccgtgttgctgtcagttccctcttagcagttggagcagtacatcaacaccttgttgcaaatcttgagaggacacgcgtaggattattggttgagtctgctgaacctcgtgaagtgcaccatttctgtacactcgttggatttggtgcagatgctatatgcccttatttggccattgaagcaatttggtgcttgcagactgatgggaagattccccctaccgactcaaaggaggaacttgtcgaaaaatatttttatgcttccatctatggaatgatgaaggttcttgcaaagatgggaatatccacccttgcatcttacaaaggggcacagatttttgaagctcttggactttcttctgaagtgattcacaagtgttttgaaggcactcctagcagaattgagggtgcaacgttcgaaatgcttgcacgtgatgctctccgtcttcacgagttggcattcccatcaagaacacctccgcctggcagtgcagatgcaaaagcccttcccaatccaggggattaccactggaggaaaaatggtgaagtccatctaaatgatcctcttgcaatggcaaaattacaagaagcagctaaagtaaacagccgggaagcatacaaagaatattctaagcgaattcaagagcttaacaaggcgtgcaatctgcgtggtatgctgaaatttatagatagcactagcaagatttctttggatgaggttgaacctgcaagtgagatagtgaaacgcttttgtactggggcaatgagttatggatctatttcgttggaggcacatactgcccttgctgtggccatgaacaaactgggaggcaaatctaacacaggggagggaggggagcagccttctcgtatggagcctcttcctgatggttcgatgaatccaaaacgaagtgcaatcaagcaagttgccagtggacgatttggagtttccagctattatctgactaatgcagatgggctgcagataaaaatggctcagggtgctaagcctggtgaaggtggtgagcttccaggtcacaaggttatcggtgacattgcagttaccagacattctacagctggtgttgggcttattagtccacctcctcaccatgatatatattctatcgaggatcttgcacagcttatccatgaccttaagaattcaaatcctcaagctcgaatcagcgtgaagctagtgtctgaggctggtgttggagttgtagctagcggtgttgtaaaaggacacgcagaccatgttcttatttctggccatgacggtggcaccggtgcatcaagatggacaggtatcaagaatgctggacttccatgggaactaggattggctgagacacatcaaactttagtggcaaatgggcttcgtggtcgagctgtcctacaaacagatggccaattaaaaactggtagagatgttgctgtggcgtgcttacttggtgcagaggaatttggtttcagcactgctccactgatcacacttggctgcattatgatgcggaagtgccataccaatacttgccctgttggtatagccactcaagatccagtgctccgagaaaaatttgctggagaaccagagcatgtcattaactttttcttcatgcttgctgaggagctaagagaaattatggctcagcttggcctccgtacaattaatgaaatggttggacgctctgatatgcttgaagttgatccagaagtagttaagagcaatgagaaacttgaaaatattgatctctcattgatcttaaaaccagctgcagagattcgtcctggggctgctcagtactgtgttgaaaagcaagaccatggccttgacatggctttggataacaaacttatagctttatcaagggctgcacttgaaaaggaagttcgtgttttcattgagactccaatcaagaacactaatcgggcagtaggcaccacacttagccatgaagtcacaaaacgttatcacatgaagggattagatcctggaaccattcatgtgaaactcactggaagtgctggtcagagttttggtgcttttctctgtcctggaataacccttgagcttgaaggagacagcaatgattacgttggcaaaggattatctggtggaaagattgttgtgtacccacccaggaacagtacatttagtgcagaagacaatattgtcattggtaatgtggccctgtatggtgctaccaagggagaagcatacttcaatggaatggcagcagaaaggttttgtgttcgtaattctggtgctcgaacagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagaggatgatgtaaccactttgaaaatgatgatagagcaacatcgacttcacacagagagtgtcctggccaaagacatactctctaaattcgacactcttcttccaaaatttgtaaaagtatacccaagggattataagagggttctagaagaaatgaaggcagaaaaagctgcagctaggcctacaaaggaacctaaggtggcaaatggtgtttctgtgacaactaagaaaatacaaacagagaagtcatccagccgaccaacacgtgttgccaacgccaaaaagtaccggggttttgtaacatatgagcgagagggtgtttcttaccgtgatccaaatgagcgtgttaaagattggaacgaggttgccattgaatcagttccagggccactgttaaacacacagtctgctcgttgtatggattgtggcactcctttctgtcatcaggaaagctcaggtgctggctgtcctcttggaaataagatcccagagttcaatgaattagttcaccagaatagatggcgtgaagcattggatcgcctactagagacaaacaacttccctgaatttactggacgtgtgtgccctgctccttgtgaggggtcttgtgttcttggcattattgagaacccagtatcaatcaaaagcatagaatgctcgattatagacaaaggttttgaagagggatggatggtaccacgaccaccacttcaaagaacaggaaagaaaatcgctatagttggcagtggtcctgctggtttggctgccgctgatcaactaaataaaatgggccattttgtaactgtatttgaacgttcggatcgtataggaggtcttatgatgtatggagtaccaaacatgaagacagacaagattggagttgttcagcgtcgtgtcaatttaatggccgaagagggtgtaacatttgtggtgaatgctaatgtagggagtgatcctttatactcaattgaacgtctccattctgagaacaatgcagttattttggcttgtggagctacaaaaccaagggacctcagtattcctggccgtgagctagctggagttcattttgccatggaatttctccacgcaaataccaaaagtttgcttgatagcaacctggaggatggaagatacatatctgcccagggtaagaaggtggtggtcattggtggtggagacacaggcacagattgcatcggtacgtctgttaggcatggttgcagcagcattgtaaatctggagcttctcaccaagccaccaagcaagagagcttctgacaacccctggccccagtggcctagagtcttccgagtggactatgggcaccaggaagcatctaccaaatttggaaatgatccaagaacttactaagtcttaaccaagcgtttcattggtgatgaagatggaaaattgaaggcccttgaggtggtgcgcgtgaagtgggagaaagtagatggaaaattccagttcaaggagattgaaggatcacaagagatcatcgaggcagaccttgtcctccttgccatgggattcctgggccctgaagagaacatcgctgacaaactgggtttagagaaagacaaccgttccaacttcaaagctcaattcggacacttcgggaccagtgtggatggcgtttttgccgctggggattgcaggcgcgggcaatcgctggttgtttgggccatcaccgaagggcgtgaagctgctgctgcagtagataagtacttgtcaagggatgaacaaaatgtcgcaggccttaca

SEQ ID NO: 6 - protein, 2167 aa, Oryza sativa
MSAAQGMAYKLRTDAAPTGAGRRARRSHSSVAAPYRAARLVQGGVSIEGGLVGGCQLTEERVAARPPRAAARDAEPVRPLSTLPESSIGLYDPSRERDSCGVGFVAELSGDYKRATVNDALEMLERMAHRGACGCEKNTGDGAGILVALPHNFFREVTKDAGFELPQPGEYAVGMVFLPIDEKRRERSKAEFQKVAESLGHVILGWRRVPTDNSDLGESALQTEPVIEQVFLTKSSSSEADFEQQLYILRRLSILSIRAALNLRRGGKRDFYMCSLSSRTIVYKGQLKPCQLKGYYYADLGHENFTSYMALVHSRFSTNTFPSWDRAQPMRVLGHNGEINTLKGNKNWMKAREGLLECEKLGLTKDQFSKILPIVDATSSDSGAFDGVLELLIRGGRSLPEAVMMMIPEAWQNDVNMEPEKKALYEFLSALMEPWDGPALISFTDGRYLGATLDRNGLRPGRFYVTHSGRVVMGSEVGVVDVPSKDVLRKGRLNPGMMLLVDFENHTVVDDEALKAQYSKAHPYGEWLKRQKIYLKDIVESVPETERVAPGISGSLTQKNEKKEHAGVNGIVTPLKAFGYTVEALEMLLLPMAKDGVEALGSMGNDTPLAVMSNREKLTFEYFKQMFAQVTNPPIDPIREKIVTSMECMIGPEGDLLETTEKQCNRLALEGPLVSIDEMEAIKKMNYRGWRSKVLDITYPKKSGRKGLEETLDRICTEARGAIKKGYTVLVLSDRGFSSDRVAVSSLLAVGAVHQHLVANLERTRVGLLVESAEPREVHHFCTLVGFGADAVCPYLAIEAIWCLQNDGKIPPNGDGKPYSKEELVKKYFYASNYGMMKVLAKMGISTLASYKGAQIFEALGLSSEVIRKCFDGTPSRIEGATFEMLARDALRLHELAFPSRAPPPGSADAKALPNPGDYHWRKNGEVHLNDPLAMAKLQEAARVNSRAAYKEYSRRIQELNKTCNLRGMLKFKDTADMISVDEVEPASEIVKRFVTGAMSYGSISLEAHTALAMAMNKLGGKSNTGEGGEQPSRMEPLANGSMNPKRSAIKQVASGRFGVSSYYLTNADELQIKMAQGAKPGEGGELPGHKVIGDIAVTRHSTAGVGLISPPPHHDIYSIEDLAQLIHDLKNSNPRARISVKLVSEAGVGVVASGVVKGHADHVLISGHDGGTGASRWTGIKNAGLPWELGLAETHQTLVANGLRGRAILQTDGQLKTGKDVAVACLLGAEEFGFSTAPLITLGCIMMRKCHTNTCPVGIATQDPVLREKFAGEPEHVINFFFMLAEELREIMSQLGFRTITEMVGRSDMLEVDPEVVKSNEKLENIDLSLILKPAAEIRPGAAQYCVEKQDHGLDMALDNKLIALSKAALEKEVRVFIETPIQNTNRAVGTMLSHEVTKRYHMKGLPAGTIHVKLTGSAGQSLGAFLCPGITLELEGDSNDYVGKGLSGGKIVVYPPRDSTFIPEDNIVIGNVALYGATIGEAYFNGMAAERFCVRNSGAQAVVEGIGDHGCEYMTGGTVVILGKTGRNFAAGMSGGIAYVYDIDGKFSVRCNHELVDLYHVEEEEDITTLKMMIEQHRLNTGSVVARDILSNFDTLLPKFVKVFPRDYKRVLDNMKAEKAAAKLAKEPKISNGVSVTTKKVQPEQSTNRPTRVSNAKKYRGFISYERESISYRDPNERVKDWKEVAIESVPGPLLNTQSARCMDCGTPFCHQESSGAGCPLGNKIPEFNELVHQNRWREALDRLLETNNFPEFTGRVCPAPCEGSCVLGIIENPVSIKSIECAIIDKGFEEGWMVPRPPLQRTGKKVAIIGSGPAGLAAADQLNKMGHFVTVFERADRIGGLMMYGVPNMKTDKIEIVQRRVNLMAEEGITFVVNANVGSDPLYSIERLRSENDAVILACGATKPRDLGIPGRELSGVHFAMEFLHANTKSLLDSNLEDGRYISAKGKKVVVIGGGDTGTDCIGTSIRHGCTSIVNLELLTKPPSKRAADNPWPQWPRIFRVDYGHQEASSKFGNDPRTYEVLTKRFIGDENGNVKALEVVRVKWEKVDGRFQFKEIEGSNETIEADLVLLAMGFLGPEATIAEKLGLEKDNRSNFKAQFGNFATSVDGIFAAGDCRRGQSLVVWAITEGRQAAAAVDKYLSRNEQDAAEDITPSGAGFVQPVAA

SEQ ID NO: 7 - DNA, 20 bp, Artificial
  Primer
ttagtggcaaatgggcttcg

SEQ ID NO: 8 - DNA, 21 bp, Artificial
  Primer
cgccacagcaacatctctacc

SEQ ID NO: 9 - DNA, 21 bp, Artificial
  Primer
cagctgcagagattcgtcctg

SEQ ID NO: 10 - DNA, 23 bp, Artificial
  Primer
tgttatccaaagccatgtcaagg

SEQ ID NO: 11 - DNA, 20 bp, Artificial
  Primer
tggaatggcagcagaaaggt

SEQ ID NO: 12 - DNA, 21 bp, Artificial
  Primer
tcgcatccatgatcaccaatt

SEQ ID NO: 13 - DNA, 23 bp, Artificial
  Primer
gcaccatttctgtacactcgttg

SEQ ID NO: 14 - DNA, 22 bp, Artificial
  Primer
atcttcccatcagtctgcaagc

SEQ ID NO: 15 - DNA, 22 bp, Artificial
  Primer
ttcaagagcttaacaaggcgtg

SEQ ID NO: 16 - DNA, 22 bp, Artificial
  Primer
cacttgcaggttcaacctcatc

SEQ ID NO: 17 - DNA, 20 bp, Artificial
  Primer
tgggaaatgatgcaccccta

SEQ ID NO: 18 - DNA, 23 bp, Artificial
  Primer
cttgtgcaaacatctgcttgaag

SEQ ID NO: 19 - DNA, 21 bp, Artificial
  Primer
aattctggaaggaagggcttg

SEQ ID NO: 20 - DNA, 23 bp, Artificial
  Primer
tttgtatccctcgcgtatagctt

SEQ ID NO: 21 - DNA, 500 bp, Artificial
  NADH-GoGAT fragment
ggggtgcagccatgccgacggcgcaggggattggtttgaagcacgcgacgccgacgggcgtcggccgcagggcccggcgcagccacgccgcgtccgcgcatggccgctccgcgaggcaggcgcacggtgccatgtctctggagggcggcgggttcctcggcggcgcgcaccgcatcgaggaacgcgtcgcgccatgcccgcctcgggccgcggcgcgtgacgcagagtcgatacggcccatgtctctgctacccgagagcagcattgggctctacaacccggcgttcgagcgtgactcgtgcggcgttggtttcgtcgccgagctgtcgggcgttgacaaccgggcgaccgtcgtcgatgccattcagatgcttgaaagaatggcacaccgaggtgcctgcggctgtgagaaaaacactggtgatggtgccggcattctcgttgctctaccacacaacttcttccgagaggtgacaaaggatgccggtttcgagttaccg

SEQ ID NO: 22 - protein, 2144 aa, Triticum aestivum
MPTAQGIGLKHAAPPGGRRARRSQSAAAPGRSARQAHGAMSLDGGFLGGAQRTEERVAPRPPRAAARDAESIRPMSLLPESSIGLYDPAFERDSCGVGFVAELSGVDNRATVVDAIQMLERMAHRGACGCEKNTGDGAGILVALPHTFFREVTKDAGFELPPPGEYAVGMVFLPTDEKRRERSKTEFTKVAESLGHSILGWRQVPTDNSDLGQAALDTEPAIEQVFLTKSSKSKADFEQQLFILRRLSIVSIRAALNLQRGGERDFYMCSLSSRTIVYKGQLMPSQLQGYYYADIGHENFSSYMALVHSRFSTNTFPSWDRAQPMRVLGHNGEINTLKGNKNWMKAREGLLECEKLGLSQDEMSKILPIVDATSSDSGAFDGVLELLIRGGRSLPEAVMMMIPEAWQNDVNMEPDKKALYEFLSALMEPWDGPALISFTDGRYLGATLDRNGLRPGRFYVTHSGRVVMGSEVGVVDIPAQDVLRKGRLNPGMMLLVDFDNHTVVDDEALKAQYSKAHPYGEWLKRQKMYLKDIVESVPETDRVAPSISGSITQTNENKECVGINAIVTPLKAFGYTLEALEMLLLPMAKDGVEALGSMGNDAPLAVMSNREKLTFEYFKQMFAQVTNPPIDPIREKIVTSMECMIGPEGDLLEITEKQCNRLALKGPLVSMDEMESIKKMNYRGWRSKVLDITYPKNSGRKGLEETLDRICAEAREAIREGYKILVLSDRGFSSDRVAVSSLLAVGAVHQHLVANLERTRVGLLVESAEPREVHHFCTLVGFGADAICPYLAIEAIWCLQTDGKIPPTDSKEELVEKYFYASIYGMMKVLAKMGISTLASYKGAQIFEALGLSSEVIHKCFEGTPSRIEGATFEMLARDALRLHELAFPSRTPPPGSADAKALPNPGDYHWRKNGEVHLNDPLAMAKLQEAAKVNSREAYKEYSKRIQELNKACNLRGMLKFIDSTSKISLDEVEPASEIVKRFCTGAMSYGSISLEAHTALAVAMNKLGGKSNTGEGGEQPSRMEPLPDGSMNPKRSAIKQVASGRFGVSSYYLTNADGLQIKMAQGAKPGEGGELPGHKVIGDIAVTRHSTAGVGLISPPPHHDIYSIEDLAQLIHDLKNSNPQARISVKLVSEAGVGVVASGVVKGHADHVLISGHDGGTGASRWTGIKNAGLPWELGLAETHQTLVANGLRGRAVLQTDGQLKTGRDVAVACLLGAEEFGFSTAPLITLGCIMMRKCHTNTCPVGIATQDPVLREKFAGEPEHVINFFFMLAEELREIMAQLGLRTINEMVGRSDMLEVDPEVVKSNEKLENIDLSLILKPAAEIRPGAAQYCVEKQDHGLDMALDNKLIALSRAALEKEVRVFIETPIKNTNRAVGTTLSHEVTKRYHMKGLDPGTIHVKLTGSAGQSFGAFLCPGITLELEGDSNDYVGKGLSGGKIVVYPPRNSTFSAEDNIVIGNVALYGATKGEAYFNGMAAERFCVRNSGARTVVEGIGDHGCEYMTGGTVVILGKTGRNFAAGMSGGIAYVYDVDGTFSVRCNNELVDLYHVEEEDDVTTLKMMIEQHRLHTESVLAKDILSKFDTLLPKFVKVYPRDYKRVLEEMKAEKAAARPTKEPKVANGVSVTTKKIQTEKSSSRPTRVANAKKYRGFVTYEREGVSYRDPNERVKDWNEVAIESVPGPLLNTQSARCMDCGTPFCHQESSGAGCPLGNKIPEFNELVHQNRWREALDRLLETNNFPEFTGRVCPAPCEGSCVLGIIENPVSIKSIECSIIDKGFEEGWMVPRPPLQRTGKKIAIVGSGPAGLAAADQLNKMGHFVTVFERSDRIGGLMMYGVPNMKTDKIGVVQRRVNLMAEEGVTFVVNANVGSDPLYSIERLHSENNAVILACGATKPRDLSIPGRELAGVHFAMEFLHANTKSLLDSNLEDGRYISAQGKKVVVIGGGDTGTDCIGTSVRHGCSSIVNLELLTKPPSKRASDNPWPQWPRVFRVDYGHQEASTKFGNDPRTYEVLTKRFIGDEDGKLKALEVVRVKWEKVDGKFQFKEIEGSQEIIEADLVLLAMGFLGPEENIADKLGLEKDNRSNFKAQFGHFGTSVDGVFAAGDCRRGQSLVVWAITEGREAAAAVDKYLSRDEQNVAGLT

SEQ ID NO: 23 - DNA, 10174 bp, Triticum aestivum
atgccgacggcgcaggggattggtttgaagcacgcggcgccgccgggcggccgcagggcccgccgcagccagtccgcggccgcgccgggccgctccgcaaggcaggcgcacggcgccatgtccctggatggcgggttcctcggcggcgcgcagcgcaccgaggaacgcgtcgcgccacgcccgcctcgggccgcggcgcgcgacgccgagtccatcaggcccatgtctctgctacccgagagcagcattgggctgtacgacccggcgttcgagcgtgactcgtgcggcgttggcttcgtcgccgagctgtcgggcgttgacaaccgggcgaccgtgagttctttgcaccaggacaccgccgcagcttctctcttttatctaccacgttgagatattgattttgctgtgatttactttacaggtcgtcgatgccattcagatgcttgaaagaatggcacaccgaggtgcctgcggctgtgagaaaaacactggtgatggtgccggcattctcgttgctctaccacacaccttcttccgagaggtgaataaacagaaaatttcaagcaaactcagttttccttgacagagttattcatcttgggtgttgctttgctattgctgcaggtgacaaaggatgccggtttcgagttaccgccaccaggtgagtatgctgttggaatggtcttcctgccaaccgatgagaagcgccgcgagaggagcaaaactgagtttacaaaggttggttggttggtgtatagacaattagacagcgtgcgaggcgagtctgcgatggtaagcttatgtcttggattgtttttattcatgcaggtcgctgagtctctaggacattcgatacttgggtggcgccaggttcccactgacaattcagacttgggccaagctgcgctcgatactgaaccagccattgaacaggttttcctcaccaagagttcaaaatcgaaggccgacttcgaacagcaggtcctccatgagtttaattttctctcaacacatgtaatgagctttcctctcaactaacccatcaattatcacagttgtttatcctgaggaggctctcaattgtatctatccgggccgcgctgaatcttcagcgtggaggagagagagatttctacatgtgctctctatcttcaaggtccttacttcttctgaattgcattttcactacaaactgatgaggttgagatcagggagcccaattaagctcctgactttaatggaacattaacaacacaaaagttgataggaaagctgactgggaaaaacaatcctgggctatctcttaagcattagaaatctgtaatgtaaggacatttcatttttgctgtattcatgtgagtaattattgttaaatcttctattgtggttttccaggaccattgtttacaagggccaacttatgccatcccagctccaagggtactactatgcggatataggtcatgaaaacttctccagttatatggctctggtaaatacgaaaccttgatactcttacaacaaaagtcatggtcgtagtcagcggatgggaatcaataatagcaagccagttgtaagatcaagactcgctcctgtgcagacatcaaatttgagctgtctattcatatgctatttcagtcctgcttcgtgatgcctttaagtgaagtactagaaacttctcttagttgatacccgataaccatgttgcagagtccagctgcagttctgctatggccgactgcttgccgactcttcaggatgtgaccgaagaaattgtgtttggtttgaggtagcttagggctcccaactgttttgcatgaaattatacgcagctgacgtatttttacaaaattcgttcttcacaaggaagttttgttttgaatatctgcaaaagatgtttataatatgatttctaaaagtcatatcttgaaacaggttcactcaaggttctccaccaacaccttccccagctgggaccgtgcacagccaatgcgtgtcttaggccacaatggagagatcaatactctcaaagggaacaaaaactggtaatattattgtcaaatcttttcctctttgctttaaccttttcttggcagatgcaactatctcaatatgcacgttgtatatatggcaggatgaaagctcgtgagggtctcttggagtgtgagaagcttggcctatcacaggatgaaatgtcaaagattcttccaatagtagatgccacttcttcagattcaggtccgcagtctaatatgcagcatgaattccacttggcaaataaaaattcatctcggttttgtgagttactaagtgtgaaaaaacatgtgaattctaggtgcatttgatggtgttctcgagctccttatccgtggtggaagaagcctgccagaagctgtgatgatgatgatccctgaggcgtggcagaatgatgtaaacatggaacctgataagaaagctctgtacgagttcttgtcagcccttatggagccttgggatggacctgctctcatatcttgtaaaaaacttaaacccgtttcttttttctgatcatacttctaactgattacttaagctaacatgggtacaacctgtagttaccgatggccgctaccttggagctaccctggatcgcaatggccttaggcctggtcgattttatgtaacccacagtggacgtgtggtcatgggtagtgaagttggtgttgtggatattcctgcccaagatgtgttgagaaagggtcggcttaaccctggaatgatgttactcgttgacttcgacaaccatactgtagtagatgatgaagcactcaaggcacagtactctaaagctcacccgtatggagaatggctcaagcgacaaaagatgtacctcaaagacattgtagaatctgtcccagaaactgacagagttgctccaagcatttctggttctattacggtaagtacttacgtggtccaacttcctcattcactgggtagtttggtgatgaaaattggtaaaccataaggaacatggtgattttaatattcttttacaatttggtgtagcaaacaaatgagaacaaggaatgtgtaggcatcaatgcaattgtgactccactaaaggcgtttgggtaagcaaggattgaacattgtcaactatcactcatgtttcaatttctgctggctaatgatccaaccgattaactacaggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgcgagggatacaaaattttagttctttcagacagaggtcagtgacttattcactttatacttgcatcccattgtttgatggtacaaattactcataagttgacaaaaaattctagcagatgggtcaggtgtgcacaatttattatcgttagcttccagcaaacatatcacatcaccatatagtaacttaaatatgatcataattttgtttgatagtcaattgtttgcagtgtaatgtgttgtatgaaggacattcttactttgtcaccttggacataattggaatgtatattaagtttattatgcaactcaacatcttccaatactgaaatttgttgtattttcttgattcaggattttcttcagaccgtgttgctgtcagttccctcttagcagttggagcagtacatcaacaccttgttgcaaatcttgagaggacacgcgtaggattattggttgagtctgctgaacctcgtgaagtgcaccatttctgtacactcgttggatttggtgcagatgctatatgcccttatttggccattgaagcaatttggtgcttgcagactgatgggaagattccccctaccgactcaaaggaggaacttgtcgaaaaatatttttatgcttccatctatggaatgatgaaggttcttgcaaagatgggaatatccacccttgcatcttacaaaggggcacagatttttgaagctcttggactttcttctgaagtgattcacaagtgttttgaaggcactcctagcagaattgagggtgcaacgttcgaaatgcttgcacgtgatgctctccgtcttcacgagttggcattcccatcaagaacacctccgcctggcagtgcagatgcaaaagcccttcccaatccaggggattaccactggaggaaaaatggtgaagtccatctaaatgatcctcttgcaatggcaaaattacaagaagcagctaaagtaaacagccgggaagcatacaaagaatattctaagcgaattcaagagcttaacaaggcgtgcaatctgcgtggtatgctgaaatttatagatagcactagcaagatttctttggatgaggttgaacctgcaagtgagatagtgaaacgcttttgtactggggcaatgagttatggatctatttcgttggaggcacatactgcccttgctgtggccatgaacaaactgggaggcaaatctaacacaggtatttcatgcatatgtgacattttttaccctttggctggttgttacccttctgatgaaatcatgtatagtagctttacttgcacatcaactcttagagccttgttgcataacatatttttcttatttttgctgcagtagtgtagtgtctatgtttctactgtttaaatatgccctcgaagttggtactttatacatgaccatgtctagaaatgcactacttattttgatgctatcacatgaaaactatactctcatatttacttgtcgacatacttgtactgcacttgtgtgtttggttgcattctcgtctcagcatagttccctcttcatgcatgctcaactcaacacccctcttcatgcatgctttcttcatgcatgcttctagtgtatttgtgctaagctagtgtggactcatgcaccctccatacactctaccaaacacccaaaagtgggtcgagaagggaactcttgggctcatgcacccttcatacaccctaccaaacacacccttaatgcgctaagcttttcatcttcctgttgatgttgaatgcacttctgtgaacaaattatcccagttcatagcaagagttaacttctaagcttttatctatctgttgatgttgaatgcacttctggtagcaaattatcctaattcctagcaagatttaacttctgcttgcgtagaaacccagacagcttgcacgcactattcttctttggactgtttgtattagatattttctgcccacacagttaatattatattgccaaatctttgtatttattttcgctcctaaactaaactcattttacccatctgctaatttaggggagggaggggagcagccttctcgtatggagcctcttcctgatggttcgatgaatccaaaacgaagtgcaatcaagcaagttgccagtggacgatttggagtttccagctattatctgactaatgcagatgggctgcagataaaaatggctcaggtacttgacaatgctgtgcattgcgcaatcactggaatgatctcagtttatagaaagcaactgtattcaacgtcctgtggttggcatttattctttttttgtatcgtatatgtatagggtgctaagcctggtgaaggtggtgagcttccaggtcacaaggttatcggtgacattgcagttaccagacattctacagctggtgttgggcttattagtccacctcctcaccatgatatatattctatcgaggatcttgcacagcttatccatgaccttaaggtacattacctgatgctcttctatagccaaggcatctgcctatttatgtatggttgttagttgctacttctagattatattggttatctaaacgctgtgatggtctaaccctcctgagaagctagaatcacttgctaaactggtacaagtgatcctgcacatatgcatgtgtctccatttgggggtttaccctcctttttgaaaaactcattttgcaaatgttacctgctgatgtagtgatattcatgcctgttttctctctgcagaattcaaatcctcaagctcgaatcagcgtgaagctagtgtctgaggctggtgttggagttgtagctagcggtgttgtaaaaggacacgcagaccatgttcttatttctggccatgacggtggcaccggtgcatcaagatggacaggtatcaagaatgctggacttccatgggaactaggattggctgagacacatcaaactttagtggcaaatgggcttcgtggtcgagctgtcctacaaacagatggccaattaaaaactggtagagatgttgctgtggcgtgcttacttggtgcagaggaatttggtttcagcactgctccactgatcacacttggctgcattatgatgcggaagtgccataccaatacttgccctgttggtatagccactcaagatccagtgctccgagaaaaatttgctggagaaccagagcatgtcattaactttttcttcatgcttgctgaggagctaagagaaattatggctcagcttggcctccgtacaattaatgaaatggttggacgctctgatatgcttgaagttgatccagaagtagttaagagcaatgagaaacttgaaaatattgatctctcattgatcttaaaaccagctgcagagattcgtcctggggctgctcagtactgtgttgaaaagcaagaccatggccttgacatggctttggataacaaacttatagctttatcaagggctgcacttgaaaaggaagttcgtgttttcattgagactccaatcaagaacactaatcgggcagtaggcaccacacttagccatgaagtcacaaaacgttatcacatgaagggattagatcctggaaccattcatgtgaaactcactggaagtgctggtcagagttttggtgcttttctctgtcctggaataacccttgagcttgaaggagacagcaatgattacgttggcaaaggattatctggtggaaagattgttgtgtacccacccaggaacagtacatttagtgcagaagacaatattgtcattggtaatgtggccctgtatggtgctaccaagggagaagcatacttcaatggaatggcagcagaaaggttttgtgttcgtaattctggtgctcgaacagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagaggatgatgtaaccactttgaaaatgatgatagagcaacatcgacttcacacagagagtgtcctggccaaagacatactctctaaattcgacactcttcttccaaaatttgtaaaagtatacccaagggattataagagggttctagaagaaatgaaggcagaaaaagctgcagctaggcctacaaaggaacctaaggtggcaaatggtgtttctgtgacaactaaggtaatatatgatttaagtaataattcttctgcatacctttttaatttgaatttatagtagagatggtgttctccatttaggtttatccatcagaaattgcaatgatacacattgccactactgtgtgaacttctatttttgcctaagtagattaactttgatctacagaaaatacaaacagagaagtcatccagccgaccaacacgtgttgccaacgccaaaaagtaccggggttttgtaacatatgagcgagagggtgtttcttaccgtgatccaaatgagcgtgttaaagattggaacgaggttgccattgaatcagttccagggccactgttaaacacacagtctgctcgttgtatggattgtggcactcctttctgtcatcaggtgaagctgagttcatcttttctatttaacattgatgattgtagttggatttattgtggggttaaggattgatatgtttagtcattgactacatatgacattgagatgctaagatttcatgattatttgtacaggaaagctcaggtgctggctgtcctcttggaaataagatcccagagttcaatgaattagttcaccagaatagatggcgtgaagcattggatcgcctactagagacaaacaacttccctgaatttactggacgtgtgtgccctgctccttgtgaggggtcttgtgttcttggcattattgagaacccagtatcaatcaaaagcatagaatgctcgattatagacaaaggttttgaagagggatggatggtaccacgaccaccacttcaaagaacagggtatgtcttttttaccatctagttcggcttattcccttatttatttacttctgtttcattaataggtaacatttgagttacactttctaatataaactgttcctttcgcagaaagaaaatcgctatagttggcagtggtcctgctggtttggctgccgctgatcaactaaataaaatgggccattttgtaactgtatttgaacgttcggatcgtataggaggtcttatgatgtatggagtaccaaacatgaagacagacaagattggagttgttcagcgtcgtgtcaatttaatggccgaagagggtgtaacatttgtggtgaatgctaatgtagggagtgatcctttatactcaattgaacgtctccattctgagaacaatgcagttattttggcttgtggagctacaaaaccaaggtaactaattggaacgaggattttttttttctagtttactccaagagtagcaacaatctcaaaagaactgacatatatcttttgacactaactgactaacatcatgtgggatattcttgaacagggacctcagtattcctggccgtgagctagctggagttcattttgccatggaatttctccacgcaaataccaaaagtttgcttgatagcaacctggaggatggaagatacatatctgcccagggtaagaaggtggtggtcattggtggtggagacacaggcacagattgcatcggtacgtctgttaggcatggttgcagcagcattgtaaatctggagcttctcaccaagccaccaagcaagagagcttctgacaacccctggccccaggtaaatgcaaaaaaaaaaaaactggatatttccctgcacatgtgaatttggccattccatttccagcttggaaagttcttaagtcatcattctgtttttgcatgctgtggcctagagtcttccgagtggactatgggcaccaggaagcatctaccaaatttggaaatgatccaagaacttacgaagtcttaaccaagcgtttcattggtgatgaagatggaaaattgaaggcccttgaggtggtgcgcgtgaagtgggagaaagtagatggaaaattccagttcaaggagattgaaggatcacaagagatcatcgaggcagaccttgtcctccttgccatgggattcctgggccctgaagaggttagttatgcagactctgaactactgtcacgcctgagttttatcttgatgataaatgttggacaaacaactcaccgttttatttctctgcagaacatcgctgacaaactgggtttagagaaagacaaccgttccaacttcaaagctcaattcggacacttcgggaccagtgtggatggcgtttttgccgctggggattgcaggcgcgggcaatcgctggttgtttgggccatcaccgaagggcgtgaagctgctgctgcagtagataagtacttgtcaagggatgaacaaaatgtcgcaggccttacatag

SEQ ID NO: 24 - DNA, 10036 bp, Triticum aestivum
  misc_feature (1985)..(1985): n is a, c, g, or t
atgtcggcggcgcaagggattggtttgaagcacgcggcgccgacgggcgtcggccgcagggcccggcgcagccacgccgcgtccgcgcatggccgctccgcgaggcaggcgcacggcgccatgtctctggagggcggcgggttcctcggcggcgcgcaccgcatcgaggaacgcgtcgcgccatgcccgcctcgggccgcggcgcgcgacgcagagtcgatacggcccatgtctctgctacccgagagcagcattgggctctacaacccggcgttcgagcgtgactcgtgcggcgttggtttcgtcgccgagctgtcgggcgttgacaaccgggcgaccgtgagttctttgcaccaaggacaccgctgcagcttctctcttccatctacagggttgagatattgatttggctgtgatttactttacaggtcgtcgatgccattcagatgcttgaaagaatggcacaccgaggtgcctgcggctgtgagaaaaacactggtgatggtgccggcattctcgttgctctaccacacaacttcttccgagaggtgaatcaacgaaaattgcaagcaaactcagttttccctgacagagttattcatcttgggtgttgctctgctattgccgcaggtgacaaaggatgccggtttcgagttaccgccaccaggtgagtatgctgttggaatggtcttcctgccaaccgatgagaagcgtcgcgagaggagcaaaactgagtttacaaaggttggttggttgctgtatagacaattagacagcgtgtgaggcgagtctgcgatgataagcttatgccttggattttttttattcacgcaggtcgcggagtcgctaggacattcgatacttgggtggcgccaggttcccactgacaattcagacttgggccaagctgctctcgacactgaaccagcgattgaacaggttttcctcaccaagagtccaaactcgaaggccgacttcgaacagcaggtcctccatgagtttaattttctctcaacacatgtaatgagctttcctctcaactaacccatcaattatcacagttgtttatcctgaggaggctctcaattgtatctatccgggccgcgctgaatcttcagcgtggaggagagagagatttctacatgtgctctctatcttcaaggtccttacttcttctgaattgcattttcactacaaactgatgaggttgagatcagggagcccaattaagctcctgactttaatggaacattaacaacacaaaagttgataggaaagctgactgggaaaaacaatcctgggctatctcttaagcattagaaatctgtaatgtaaggacatttcatttttgctgtattcatgtgagtaattattgttaaatcttctattgtggttttccaggaccattgtttacaagggccaacttatgccatcccagctccaagggtactactatgcggatataggtcatgaaaacttctccagttatatggctctggtaaatacgaaaccttgatactcttacaacaaaagtcatggtcgtagtcagcggatgggaatcaataatagcaagccagttgtaagatcaagactcgctcctgtgcagacatcaaatttgagctgtctattcatatgctatttcagtcctgcttcgtgatgcctttaagtgaagtactagaaacttctcttagttgatacccgataaccatgttgcagagtccagctgcagttctgctatggccgactgcttgccgactcttcaggatgtgaccgaagaaattgtgtttggtttgaggtagcttagggctcccaactgttttgcatgaaattatacgcagctgacgtatttttacaaaattcgttcttcacaaggaagttttgttttgaatatctgcaaaagatgtttataatatgatttctaaaagtcatatcttgaaacaggttcactcaaggttctccaccaacaccttccccagctgggaccntgcacagccaatgcgtgtcttaggccacaatggagagatcaatacnctcaaagggaacaaaaactggtaatattattgtcaaatcttttcctctttgctttaaccttttcttggcagatgcaactatctcaatatgcacgttgtatatatggcaggatgaaagctcgtgagggtctcttggagtgtgagaagcttggcctatcacaggatgaaatgtcaaagattcttccaatagtngatgccacttcttcagattcaggtccgcagtctaatatgcagcatgaattccacttggcaaataaaaattcatctcggttttgtgagttactaagtgtgaaaaaacatgtgaattctaggtgcatttgatggtgttctcgagctccttatccgtggtggaagaagcctgccagaagctgtgatgatgatgatccctgaggcgtggcagaatgatgtaaacatggaacctgataagaaagctctgtacgagttcttgtcagcccttatggagccttgggatggacctgctctcatatcttgtaaaaaacttaaacccgtttcttttttctgatcatacttctaactgattacttaagctaacatgggtacaacctgtagttaccgatggccgctaccttggagctaccctggatcgcaatggccttaggcctggtcgattttatgtgacccacagtggacgtgtggtcatgggtagtgaagttggtgttgtggatattcctgcccaagatgtgttgagaaagggtcggcttaaccctggaatgatgttactcgttgacttcgacaaccatactgtagtagatgatgaagcactcaaggcacagtactctaaagctcacccgtatggagaatggctcaaacgacaaaagatgtacctcaaagacattgtagaatctgtcccagaaactgacagagttgctccaagcatttctggttctattacggtaagtacttacgtggtccaacttcctcattcactgggtagtttggtgacgaaaattggtaaaccataagtaacatggtgattttaatattcttttacaatttggtgtagcaaacaaatgagaacaaggaatgtgtaggcatcaatgcaattgtgactccactaaaggcgtttgggtaagcaaggattaaacattgtcaactatcactcatgtttcagtttctgctggctaatgatccaaccgattaactacaggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgngagggatacaaaattttagttctttcagacagaggtcagtgacttattcactttatacttgcatcccattgtttgatggtacaaattactcataagttgacaaaaaattctagcagatgggtcaggtgtgcacaatttattatcgttagcttccagcaaacatatcacatcaccatatagtaacttaaatatgatcataattttgtttgatagtcaattgtttgcagtgtaatgtgttgtatgaaggacattcttactttgtcaccttggacataattggaatgtatattaagtttattatgcaactcaacatcttccaatactgaaatttgttgtattttcttgattcaggattttcttcagaccgtgttgctgtcagttccctcttagcagttggagcagtacatcaacaccttgttgcaaatcttgagaggacacgcgtaggattattggttgagtctgctgaacctcgtgaagtgcaccatttctgtacactcgttggatttggtgcagatgctatatgcccttatttggccattgaagcaatttggtgcttgcagactgatgggaagattccccctaccgactcaaaggaggaacttgtcgaaaaatatttttatgcttccatctatggaatgatgaaggttcttgcaaagatgggaatatccacccttgcatcttacaaaggggcacagatttttgaagctcttggactttcttctgaagtgattcacaagtgttttgaaggcactcctagcagaattgagggtgcaacgttcgaaatgcttgcacgtgatgctctccgtcttcacgagttggcattcccatcaagaacacctccgcctggcagtgcagatgcaaaagcccttcccaatccaggggattaccactggaggaaaaatggtgaagtccanctaaatgatcctcttgcaatggcaaaattacaagaagcagctaaagtaaacagccgggaagcatacaaagaatattctaagcgaattcaagagcttaacaaggcgtgcaatctgcgtggtatgctgaaatttatagatagcactagcaagatttctttggatgaggttgaacctgcaagtgagatagtgaaacgcttttgtactggggcaatgagttatggatctatttcgttggaggcacatactgcccttgctgtggccatgaacaaactgggaggcaaatctaacacaggtatttcatgcatatgtgacattttttaccctttcgctggttgttacccttctgatgaaatcatgtatagtagctttacttgcacatcaactcttagagccttgttgcataacatatttttcttatttttgctgcagtagtgtagtgtctatgtttctactgtttaaatatgccctcgaagttggtactttatacatgaccatgtctagaaatgcactacttattttgatgctatcacatgaaaactatactctcatagcagtacaagtatttatcttcctgttgatgttgaatgcacttctgtgaaaactatactctcatagtagtgtctatgtttctacaagtatttatcacatgaaaactatactcacataaaaccatttcatcttcctgttgatgttgaatgcacttctgtgaacaaattatcccagttcatagcaagagttaacttctaagcttttatctatctgttgatgttgaatgcacttctggtagcaaattatcctaattcctagcaagatttaacttctgcttgcgtagaaacccagacagcttgcacgcattattcttctttggactgtttgtattatagatattttctgcccacacagttaatattatattgccaaatctttgtatttattttcactcctaaactaaactcattttacccatctgctaatttaggggagggaggggagcagccttctcgtatggagcctcttcctgatggttcgatgaatccaaaacgaagtgcaatcaagcaagttgccagtggacgatttggagtttccagctattatctgactaatgcagatgagctgcagataaaaatggctcaggtacttgacaatgctgtgcattgcgcaatcactggaatgatctcagtttatagaaagcaactgtattcaacgtcctgtggttggcatttattctttttttgtatcgtatatgtatagggtgctaagcctggtgaaggtggtgagcttccaggtcacaaggttatcggtgacattgcagttaccagacattctacagctggtgttgggcttattagtccacctcctcaccatgatatatattctatcgaggatcttgcacagcttatccatgaccttaaggtacattacctgatgctcttctatagccaaggcatctgcctatttatgtatggttgttagttgctacttctagattatattggttatctaaacgctgtgatggtctaaccctcctgagaagctagaatcacttgctaaactggtacaagtgatcctgcacatatgcatgtgtctccatttgggggtttaccctcctttttgaaaaactcattttgcaaatgttacctgctgatgtagtgatattcatgcctgttttctctctgcagaattcaaatcctcaagctcgaatcagcgtgaagctagtgtctgaggctggtgttggagttgtagctagcggtgttgtaaaaggacacgcagaccatgttcttatttctggccatgacggtggcaccggtgcatcaagatggacaggtatcaagaatgctggacttccatggggaactaggattggctgagacacatcaaactttagtggcaaatgggcttcgtggtcgagctgtcctacaaacagatggccaattaaaaactggtagagatgttgctgtggcgtgcttacttggtgcagaggaatttggtttcagcactgctccactgatcacacttggctgcattatgatgcggaagtgccataccaatacttgccctgttggtatagccactcaagatccagtgctccgagaaaaatttgctggagaaccagagcatgtcattaactttttcttcatgcttgctgaggagctaagagaaattatggctcagcttggcctccgtacaattaatgaaatggttggacgctctgatatgcttgaagttgatccagaagtagttaagagcaatgagaaacttgaaaatattgatctctcattgatcttaaaaccagctgcagagattcgtcctggggctgctcagtactgtgttgaaaagcaagaccatggccttgacatggctttggataacaaacttatagctttatcaaggnctgcacttgaaaaggaagttcgtgttttcnttgagactccaatcaagaacactaatcgggcagtaggcacnacacttagccatgaagtcacaaaacgttatcacatgaagggattagatcctggaaccattcatgtgaaactcactggaagtgctggtcagagttttggtgcttttctctgtcctggaataacccttgagcttgaaggagacagcaatgattacgttggcaaaggattatctggtggaaagattgttgtgtacccacccaggaacagtacatttagtgcagaagacaatattgtcattggtaatgtggccctgtatggtgctaccaagggagaagcatacttcaatggaatggcagcagaaaggttttgtgttcgtaattctggtgctcggacagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagaggatgatgtaaccactttgaaaatgatgatagagcaacatcgacttcacacagagagtgtcctggccaaagacatactctctaaattcgacactcttcttccaaaatttgtaaaagtatacccaagggattataagagggttctagaagaaatgaaggcagaaaaagctgcagctaggcctacaaaggaacctaaggtggcaaatggtgtttctgtgacaactaaggtaatatatgatttaagtaataattcttctgcatacctttttaatttgaatttatagtagagatggtgttctccatttaggtttatccatcagaaattgcaatgatacacattgccactactgtgtgaacttctatttttgcctaagtagattaactttgatctacagaaaatacaaacagagaagtcatccagccgaccaacacgtgttgccaacgccaaaaagtaccggggttttgtaacatatgagcgagagggtgtttcttaccgtgatccaaatgagcgtgttaaagattggaacgaggttgccattgaatcagttccagggccactgttaaacacacagtctgctcgttgtatggattgtggcactcctttctgtcatcaggtgaagctgagttcatcttttctatttaacattgatgattgtagttggatttattgtggggttaaggattgatatgtttagtcattggctacatatgacattgagatgctaagatttcatgattatttgtacaggaaagctcaggtgctggctgtcctcttggaaataagatcccagagttcaatgaattagttcaccagaatagatggcgtgaagcattggatcgcctactagagacaaacaacttccctgaatttactggacgtgtgtgccctgctccttgtgaggggtcttgtgttcttggcattattgagaacccagtatcaatcaaaagcatagaatgctcgattatagacaaaggttttgaagagggatggatggtaccacgaccaccacttcaaagaacagggtatgtcttttttaccatctagttcggcttattcccttatttatttacttctgtttcattaataggtaacatttgagttacactttctaatataaactgttcctttcgcagaaagaaaatcgctatagttggcagtggtcctgctggtttggctgccgctgatcaactaaataaaatgggccattttgtaactgtatttgaacgttcggatcgtataggaggtcttatgatgtatggagtaccaaacatgaagacagacaagattggagttgttcagcgtcgtgtcaatttaatggccgaagagggtataacatttgtggtgaatgctaatgtagggagtgatcctttatactcaattgaacgtctccgttctgagaacaatgcagttattttggcttgtggagctacaaaaccaaggtaactaattggaacgaggattttttttttctagtttactccaagagtagcaacaatctcaaaagaactgacatatatcttttgacactaactgactaacatcatgtgggatattcttgaacagggacctcagtattcctggccgtgagctagctggagttcattttgccatggaatttctccacgcaaataccaaaagtttgcttgatagcaacctggaggatggaagatacatatctgcccagggtaagaaggtggtggtcattggtggtggagacacaggcacagattgcatcggtacgtctgttaggcatggttgcagcagcattgtaaatctggagcttctcaccaagccaccaagcaagagagcttctgacaacccctggccccaggtaaatgcaaaaaaaaaaaactggatatttccctgcacatgtgaatttggccattccatttccagcttggaaagttcttaagtcatcattctgtttttgcatgcagtggcctagagtcttccgagtggactatgggcaccaggaagcatctaccaagtttggaaatgatccaagaacttacgaagtcttaaccaagcgtttcattggtgatgaagatggaaaattgaaggcccttgaggtggtgcgcgtgaagtgggagaaagtagatggaaaattccagttcaaggagattgaaggatcacaagagatcatcgaggcagaccttgtcctccttgccatgggattcctgggccctgaagaggttagttatgcagactctgaactactgtcacgcctgagttttatcttgatgataaatgttggacaaacaactcaccgttttatttctctgcagaacatcgctgacaaactgggtttagagaaagacaaccgttccaacttcaaagctcaattcggacacttcgggaccagtgtggatggcgtttttgccgctggggattgcaggcgcgggcaatcgctggttgtttgggccatcaccgaagggcgtgaagctgctgctgcagtagataagtacttgtcaagggatgaacaaaatgtcgcaggccttacatag

SEQ ID NO: 25 - DNA, 10180 bp, Triticum aestivum
  misc_feature (2207)..(2207): n is a, c, g, or t
atgtcggcggcgcaagggattggtttgaagcacgcggcgccgacgggcgtcggccgcagggcccggcgcagccacgccgcgtccgcgcatggccgctccgcgaggcaggcgcacggcgccatgtctctggagggcggcgggttcctcggcggcgcgcaccgcattgaggaacgcgtcgcgccatgcccgcctcgggccgcggcgcgcgacgcagagtccatacggcccatgtctctgctacccgagagcagcattgggctctacaacccggcgttcgagcgtgactcgtgcggcgttggtttcgtcgccgagctgtcgggcgttgacaaccgggcgaccgtgagttctttgcaccaaggacaccgctgcagcttctctcttccatctacagggttgagatattgatttggctgtgatttactttacaggtcgtcgatgccattcagatgcttgaaagaatggcacaccgaggtgcctgcggctgtgagaaaaacactggtgatggtgccggcattctcgttgctctaccacacaacttcttccgagaggtgaatcaatgaaaattgcaagcaaactcagttttccttgacagagttattcatcttgggtgtcgctctgctattgccgcaggtgacaaaggatgccggtttcgagttaccgccaccaggtgagtatgctgttggaatggtcttcctgccaaccgatgagaagcgccgcgagaggagcaaaactgagtttacaaaggttggttggttggtgtatagacaattagacagcgtgcgaggcgagtctgcgatggtaagcttatgtgttggattttttttattcatgcaggtcgctgagtcgctaggacattcgatacttgggtggcgccaggttcccactgacaattcagacttgggccaagctgcgctcgacactgaaccagcgattgaacaggttttcctcacaaagagttcaaaatcgaaggccgacttcgaacagcaggttctccatgagtttaattttctctcaacacatgtaatgagctttcctctcaactaacccatcaattatcacagttgtttatcctgaggaggctctcaattgtatctatccgggccgcgctgaatcttcagcgtggaggagagagagatttctacatgtgctctctatcttcaaggtccttacttcttctgaattgcattttcactacaaactgatgaggttgagatcagggagcccaattaagctcctgactttaatggaacattaacaacacaaaagttgataggaaagctgactgggaaaaacaatcctgggctatctcttaagcattagaaatctgtaatgtaaggacatttcatttttgctgtattcatgtgagtaattattgttaaatcttctattgtggttttccaggaccattgtttacaagggccaacttatgccatcccagctccaagggtactactatgcggatataggtcatgaaaacttctccagttatatggctctggtaaatacgaaaccttgatactcttacaacaaaagtcatggtcgtagtcagcggatgggaatcaataatagcaagccagttgtaagatcaagactcgctcctgtgcagacatcaaatttgagctgtctattcatatgctatttcagtcctgcttcgtgatgcctttaagtgaagtactagaaacttctcttagttgatacccgataaccatgttgcagagtccagctgcagttctgctatggccgactgcttgccgactcttcaggatgtgaccgaagaaattgtgtttggtttgaggtagcttagggctcccaactgttttgcatgaaattatacgcagctgacgtatttttacaaaattcgttcttcacaaggaagttttgttttgaatatctgcaaaagatgtttataatatgatttctaaaagtcatatcttgaaacaggttcactcaaggttctccaccaacaccttccccagctgggaccgtgcacagccaatgcgtgtcttaggccacaatggagagatcaatactctcaaagggaacaaaaactggtaatattattgtcaaatcttttcctctttgctttaaccttttcttggcagatgcaactatctcaatatgcacgttgtatatatggcaggatgaaagctcgtgagggtctcttggagtgtgagaagcttggcctatcacaggatgaaatgtcaaanattcttccaatagtngatgccacttcttcagattcaggtccgcagtctaatatgcagcatgaattccacttggcaaatanaaattcatctcggttttgngagttactaagtgtgaaaaaacatgtgaattctaggtgcatttgatggtgttctngagctccttatccgtggtggaagaagcctgccagaagctgtgatgatgatgatcccngaggcgtggcagaatgatgtaaacatggaacctgataagaaagctctgtacgagttcttgtcagcccttatggagccttgggatggacctgctctcatatcttgtaaaaaacttaaacccgtttcttttttctgatcatacttctaactgattacttaagctaacatgggtacaacctgtagttaccgatggccgctaccttggagctaccctggatcgcaatggccttaggcctggtcgattttatgtaacccacagtggacgtgtggtcatgggtagtgaagttggtgttgtggatattcctgcccaagatgtgttgagaaagggtcggcttaaccctggaatgatgttactcgttgacttcgacgaccatactgtagtagatgatgaagcactcaaggcacagtactctaaagctcacccgtatggagaatggctcaagcgacaaaagatgtacctcaaagacattgtagaatctgtcccagaaactgacagagttgctccaagcatttctggttctattacggtaagtacttacgtggtccaacttcctcattcactgggtagtttggtgatgaaaattggtaaaccataaggaacatggtgattttaatattcttttacaatttggtgtagcaaacaaatgagaacaaggaatgtgtaggcatcaatgcaattgtgactccactaaaggcgtttgggtaagcaaggattgaacattgtcaactatcactcatgtttcaatttctgctggctaatgatccaaccgattaactacaggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgcaagggatacaaaattttagttctttcagacagaggtcagtgacttattcactttatacttgcatcccattgtttgatggtncaaattactcataagttgacaaaaaattctagcagatgggtcaggtgtgcacaatttattatcgttagcttccagcaaacatatcacatcaccatatantaacttaaatatgatcataattttntttgatagtcaattgtttncagtgtaatgtgttgtatgaaggacattcttactttgtcaccttggacataattggaatgtatattaagtttattatgcaactcaacatcttccaatactgaaatttgttgtattttcttgattcaggattttcttcagaccgtgttgctgtcagttccctcttagcagttggagcagtacatcaacaccttgttgcaaatcttgagaggacacgcgtaggattattggttgagtctgctgaacctcgtgaagtgcaccatttctgtacactcgttggatttggtgcagatgctatatgcccttatttggccattgaagcaatttggtgcttgcagactgatgggaagattccccctaccgactcaaaggaggaacttgtcgaaaaatatttttatgcttccatctatggaatgatgaaggttcttgcaaagatgggaatatccacccttgcatcttacaaaggggcacagatttttgaagctcttggactttcttctgaagtgattcacaagtgttttgaaggcactcctagcagaattgagggtgcaacgttcgaaatgcttgcacgtgatgctctccgtcttcacgagttggcattcccatcaagaacacctccgcctggcagtgcagatgcaaaagcccttcccaatccaggggattaccactggaggaaaaatggtgaagtccatctaaatgatcctcttgcaatggcaaaattacaagaagcagctaaagtaaacagccgggaagcatacaaagaatattctaagcgaattcaagagcttaacaaggcgtgcaatctgcgtggtatgctgaaatttatagatagcactagcaagatttctttggatgaggttgaacctgcaagtgagatagtgaaacgcttttgtactggggcaatgagttatggatctatttcgttggaggcacatactgcccttgctgtggccatgaacaaactgggaggcaaatctaacacaggtatttcatgcatatgtgacattttttaccctttggctggttgttacccttctgatgaaatcatgtatagtagctttacttgcacatcaactcttagagccttgttgcataacatatttttcttatttttgctgcagtagtgtagtgtctatgtttctactgtttaaatatgccctcgaagttggtactttatacatgaccatgtctagaaatgcactacttattttgatgctatcacatgaaaactatactctcatatttacttgtggacatacttgtactgcacttgtgtgtttggttgcattctcgtctcagcatagttccctcttcatgcatgctcaactcaacacccctcttcatgcatgctttcttcatgcatgcttctagtgtatttgtgctaagctagtgtggactcatgcaccctccatacactctaccaaacacccaaaagtgggtcgagaagggaactcttgggctcatgcacccttcatacaccctaccaaacacacccttaatgcgctaagcttttcatcttcctgttgatgttgaatgcacttctgtgaacaaattatcccagttcatagcaagagttaacttctaagcttttatctatctgttgatgttgaatgcacttctggtagcaaattatcctnattcntagcaagatttaacttctgcttgcgtagaaacccagacagcttgcacgcactattcttctttggactgtttgtattagatattttctgcccacacagttaatattatattgccaaatctttgtatttattttcgctcctaaactaaactcattttacccatctgctaatttaggggagggaggggagcagccttctcgtatggagcctcttcctgatggttcgatgaatccaaaacgaagtgcaatcaagcaagttgccagtggacgatttggagtttccagctattatctgactaatgcagatgagctgcagataaaaatggctcaggtacttgacaatgctgtgcattgcgcaatcactggaatgatctcagtttatagaaagcaactgtattcaacgtcctgtggttggcatttattctttttttgtatcgtatatgtatagggtgctaagcctggtgaaggtggtgagcttccaggtcacaaggttatcggtgacattgcagttaccagacattctacagctggtgttgggcttattagtccacctcctcaccatgatatatattctatcgaggatcttgcacagcttatccatgaccttaaggtacattacctgatgctcttctatagccaaggcatctgcctatttatgtatggttgttagttgctacttctagattatattggttatctaaacgctgtgatggtctaaccctcctgagaagctagaatcacttgctaaactggtacaagtgatcctgcacatatgcatgtgtctccatttgggggtttaccctcctttttgaaaaactcattttgcaaatgttacctgctgatgtagtgatattcatgcctgttttctctctgcagaattcaaatcctcaagctcgaatcagcgtgaagctagtgtctgaggctggtgttggagttgtagctagcggtgttgtaaaaggacacgcagaccatgttcttatttctggccatgacggtggcaccggtgcatcaagatggacaggtatcaagaatgctggacttccatggggaactaggattggctgagacacatcaaactttagtggcaaatgggcttcgtggtcgagctgtcctacaaacagatggccaattaaaaactggtagagatgttgctgtggcgtgcttacttggtgcagaggaatttggtttcagcactgctccactgatcacacttggctgcattatgatgcggaagtgccataccaatacttgccctgttggtatagccactcaagatccagtgctccgagaaaaatttgctggagaaccagagcatgtcattaactttttcttcatgcttgctgaggagctaagagaaattatggctcagcttggcctccgtacaattaatgaaatggttggacgctctgatatgcttgaagttgatccagaagtagttaagagcaatgagaaacttgaaaatattgatctctcattgatcttaaaaccagctgcagagattcgtcctggggctgctcagtactgtgttgaaaagcaagaccatgccttgacatggctttggataacaaacttatagctttatcaaggnctgcacttgaaaaggaagttcgtgttttcnttgagactccaatcaagaacactaatcgggcagtaggcacnacacttagccatgaagtcacaaaacgttatcacatgaagggattagatcctggaaccattcatgtgaaactcactggaagtgctggtcagagttttggtgcttttctctgtcctggaataacccttgagcttgaaggagacagcaatgattacgttggcaaaggattatctggtggaaagattgttgtgtacccacccaggaacagtacatttagtgcagaagacaatattgtcattggtaatgtggccctgtatggtgctaccaagggagaagcatacttcaatggaatggcagcagaaaggttttgtgttcgtaattctggtgctcgaacagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagaggatgatgtaaccactttgaaaatgatgatagagcaacatcgacttcacacagagagtgtcctggccaaagacatactctctaaattcgacactcttcttccaaaatttgtaaaagtatacccaagggattataagagggttctagaagaaatgaaggcagaaaaagctgcagctaggcctacaaaggaacctaaggtggcaaatggtgtttctgtgacaactaaggtaatatatgatttaagtaataattcttctgcatacctttttaatttgaatttatagtagagatggtgttctccatttaggtttatccatcagaaattgcaatgatacacattgccactactgtgtgaacttctatttttgcctaagtagattaactttgatctacagaaaatacaaacagagaagtcatccagccgaccaacacgtgttgccaacgccaaaaagtaccggggttttgtaacatatgagcgagagggtgtttcttaccgtgatccaaatgagcgtgttaaagattggaacgaggttgccattgaatcagttccagggccactgttaaacacacagtctgctcgttgtatggattgtggcactcctttctgtcatcaggtgaagctgagttcatcttttctatttaacattgatgattgtagttggatttattgtggggttaaggattgatatgtttagtcattggctacatatgacattgagatgctaagatttcatgattatttgtacaggaaagctcaggtgctggctgtcctcttggaaataagatcccagagttcaatgaattagttcaccagaatagatggcgtgaagcattggatcgcctactagagacaaacaacttccctgaatttactggacgtgtgtgccctgctccttgtgaggggtcttgtgttcttggcattattgagaacccagtatcaatcaaaagcatagaatgctcgattatagacaaaggttttgaagagggatggatggtaccacgaccaccacttcaaagaacagggtatgtcttttttaccatctagttcggcttattcccttatttatttacttctgtttcattaataggtaacatttgagttacactttctaatataaactgttcctttcgcagaaagaaaatcgctatagttggcagtggtcctgctggtttggctgccgctgatcaactaaataaaatgggccattttgtaactgtatttgaacgttcggatcgtataggaggtcttatgatgtatggagtaccaaacatgaagacagacaagattggagttgttcagcgtcgtgtcaatttaatggccgaagagggtntaacatttgtggtgaatgctaatntagggagtgatcctttatactcaattgaacgtctccgttctgagaacaatgcagttattttggcttgtggagctacaaaaccaaggtaactaattggaacgaggattttttttttctagtttactccaagagtagcaacaatctcaaaagaactgncatatatcttttgacactaactgactaacatcatgtgggatattcttgaacagggacctcagtattcctggncgtgagctagctggagttcattttgccatggaatttctccacgcaaataccaaaagtttgcttgatagcaacctggaggatggaagatacatatctgcccagggtaagaaggtggtggtcattggtggtggagacacaggcacagattgcatcggtacgtctgttaggcatggttgcagcagcattgtaaatctggagcttctcaccaagccaccaagcaagagagcttctgacaacccctggccccaggtaaatgcaaaaaaaaaaaaactggatatttccctgcacatgtgaatttggccattccatttccagcttggaaagttcttaagtcatcattctgtttttgcatgcagtggcctagagtcttccgagtggactatgggcaccaggaagcatctaccaagtttggaaatgatccaagaacttacgaagtcttaaccaagcgtttcattggtgatgaagatggaaaattgaaggcccttgaggtggtgcgcgtgaagtgggagaaagtagatggaaaattccagttcaaggagattgaaggatcacaagagatcatcgaggcagaccttgtcctccttgccatgggattcctgggccctgaagaggttagttatgcagactctgaactactgtcacgcctgagttttatcttgatgataaatgttggacaaacaactcaccgttttatttctctgcagaacatcgctgacaaactgggtttagagaaagacaaccgttccaacttcaaagctcaattcggacacttcgggaccagtgtggatggcgtttttgccgctggggattgcaggcgcgggcaatcgctggttgtttgggccatcaccgaagggcgtgaagctgctgctgcagtagataagtacttgtcaagggatgaacaaaatgtcgcaggccttacatag

SEQ ID NO: 26 - DNA, 6435 bp, Triticum aestivum
atgccgacggcgcaggggattggtttgaagcacgcggcgccgccgggcggccgcagggcccgccgcagccagtccgcggccgcgccgggccgctccgcaaggcaggcgcacggcgccatgtccctggatggcgggttcctcggcggcgcgcagcgcaccgaggaacgcgtcgcgccacgcccgcctcgggccgcggcgcgcgacgccgagtccatcaggcccatgtctctgctacccgagagcagcattgggctgtacgacccggcgttcgagcgtgactcgtgcggcgttggcttcgtcgccgagctgtcgggcgttgacaaccgggcgaccgtcgtcgatgccattcagatgcttgaaagaatggcacaccgaggtgcctgcggctgtgagaaaaacactggtgatggtgccggcattctcgttgctctaccacacaccttcttccgagaggtgacaaaggatgccggtttcgagttaccgccaccaggtgagtatgctgttggaatggtcttcctgccaaccgatgagaagcgccgcgagaggagcaaaactgagtttacaaaggtcgctgagtctctaggacattcgatacttgggtggcgccaggttcccactgacaattcagacttgggccaagctgcgctcgatactgaaccagccattgaacaggttttcctcaccaagagttcaaaatcgaaggccgacttcgaacagcagttgtttatcctgaggaggctctcaattgtatctatccgggccgcgctgaatcttcagcgtggaggagagagagatttctacatgtgctctctatcttcaaggaccattgtttacaagggccaacttatgccatcccagctccaagggtactactatgcggatataggtcatgaaaacttctccagttatatggctctggttcactcaaggttctccaccaacaccttccccagctgggaccgtgcacagccaatgcgtgtcttaggccacaatggagagatcaatactctcaaagggaacaaaaactggatgaaagctcgtgagggtctcttggagtgtgagaagcttggcctatcacaggatgaaatgtcaaagattcttccaatagtagatgccacttcttcagattcaggtgcatttgatggtgttctcgagctccttatccgtggtggaagaagcctgccagaagctgtgatgatgatgatccctgaggcgtggcagaatgatgtaaacatggaacctgataagaaagctctgtacgagttcttgtcagcccttatggagccttgggatggacctgctctcatatcttttaccgatggccgctaccttggagctaccctggatcgcaatggccttaggcctggtcgattttatgtaacccacagtggacgtgtggtcatgggtagtgaagttggtgttgtggatattcctgcccaagatgtgttgagaaagggtcggcttaaccctggaatgatgttactcgttgacttcgacaaccatactgtagtagatgatgaagcactcaaggcacagtactctaaagctcacccgtatggagaatggctcaagcgacaaaagatgtacctcaaagacattgtagaatctgtcccagaaactgacagagttgctccaagcatttctggttctattacgcaaacaaatgagaacaaggaatgtgtaggcatcaatgcaattgtgactccactaaaggcgtttgggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgcgagggatacaaaattttagttctttcagacagaggattttcttcagaccgtgttgctgtcagttccctcttagcagttggagcagtacatcaacaccttgttgcaaatcttgagaggacacgcgtaggattattggttgagtctgctgaacctcgtgaagtgcaccatttctgtacactcgttggatttggtgcagatgctatatgcccttatttggccattgaagcaatttggtgcttgcagactgatgggaagattccccctaccgactcaaaggaggaacttgtcgaaaaatatttttatgcttccatctatggaatgatgaaggttcttgcaaagatgggaatatccacccttgcatcttacaaaggggcacagatttttgaagctcttggactttcttctgaagtgattcacaagtgttttgaaggcactcctagcagaattgagggtgcaacgttcgaaatgcttgcacgtgatgctctccgtcttcacgagttggcattcccatcaagaacacctccgcctggcagtgcagatgcaaaagcccttcccaatccaggggattaccactggaggaaaaatggtgaagtccatctaaatgatcctcttgcaatggcaaaattacaagaagcagctaaagtaaacagccgggaagcatacaaagaatattctaagcgaattcaagagcttaacaaggcgtgcaatctgcgtggtatgctgaaatttatagatagcactagcaagatttctttggatgaggttgaacctgcaagtgagatagtgaaacgcttttgtactggggcaatgagttatggatctatttcgttggaggcacatactgcccttgctgtggccatgaacaaactgggaggcaaatctaacacaggggagggaggggagcagccttctcgtatggagcctcttcctgatggttcgatgaatccaaaacgaagtgcaatcaagcaagttgccagtggacgatttggagtttccagctattatctgactaatgcagatgggctgcagataaaaatggctcagggtgctaagcctggtgaaggtggtgagcttccaggtcacaaggttatcggtgacattgcagttaccagacattctacagctggtgttgggcttattagtccacctcctcaccatgatatatattctatcgaggatcttgcacagcttatccatgaccttaagaattcaaatcctcaagctcgaatcagcgtgaagctagtgtctgaggctggtgttggagttgtagctagcggtgttgtaaaaggacacgcagaccatgttcttatttctggccatgacggtggcaccggtgcatcaagatggacaggtatcaagaatgctggacttccatgggaactaggattggctgagacacatcaaactttagtggcaaatgggcttcgtggtcgagctgtcctacaaacagatggccaattaaaaactggtagagatgttgctgtggcgtgcttacttggtgcagaggaatttggtttcagcactgctccactgatcacacttggctgcattatgatgcggaagtgccataccaatacttgccctgttggtatagccactcaagatccagtgctccgagaaaaatttgctggagaaccagagcatgtcattaactttttcttcatgcttgctgaggagctaagagaaattatggctcagcttggcctccgtacaattaatgaaatggttggacgctctgatatgcttgaagttgatccagaagtagttaagagcaatgagaaacttgaaaatattgatctctcattgatcttaaaaccagctgcagagattcgtcctggggctgctcagtactgtgttgaaaagcaagaccatggccttgacatggctttggataacaaacttatagctttatcaagggctgcacttgaaaaggaagttcgtgttttcattgagactccaatcaagaacactaatcgggcagtaggcaccacacttagccatgaagtcacaaaacgttatcacatgaagggattagatcctggaaccattcatgtgaaactcactggaagtgctggtcagagttttggtgcttttctctgtcctggaataacccttgagcttgaaggagacagcaatgattacgttggcaaaggattatctggtggaaagattgttgtgtacccacccaggaacagtacatttagtgcagaagacaatattgtcattggtaatgtggccctgtatggtgctaccaagggagaagcatacttcaatggaatggcagcagaaaggttttgtgttcgtaattctggtgctcgaacagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagaggatgatgtaaccactttgaaaatgatgatagagcaacatcgacttcacacagagagtgtcctggccaaagacatactctctaaattcgacactcttcttccaaaatttgtaaaagtatacccaagggattataagagggttctagaagaaatgaaggcagaaaaagctgcagctaggcctacaaaggaacctaaggtggcaaatggtgtttctgtgacaactaagaaaatacaaacagagaagtcatccagccgaccaacacgtgttgccaacgccaaaaagtaccggggttttgtaacatatgagcgagagggtgtttcttaccgtgatccaaatgagcgtgttaaagattggaacgaggttgccattgaatcagttccagggccactgttaaacacacagtctgctcgttgtatggattgtggcactcctttctgtcatcaggaaagctcaggtgctggctgtcctcttggaaataagatcccagagttcaatgaattagttcaccagaatagatggcgtgaagcattggatcgcctactagagacaaacaacttccctgaatttactggacgtgtgtgccctgctccttgtgaggggtcttgtgttcttggcattattgagaacccagtatcaatcaaaagcatagaatgctcgattatagacaaaggttttgaagagggatggatggtaccacgaccaccacttcaaagaacaggaaagaaaatcgctatagttggcagtggtcctgctggtttggctgccgctgatcaactaaataaaatgggccattttgtaactgtatttgaacgttcggatcgtataggaggtcttatgatgtatggagtaccaaacatgaagacagacaagattggagttgttcagcgtcgtgtcaatttaatggccgaagagggtgtaacatttgtggtgaatgctaatgtagggagtgatcctttatactcaattgaacgtctccattctgagaacaatgcagttattttggcttgtggagctacaaaaccaagggacctcagtattcctggccgtgagctagctggagttcattttgccatggaatttctccacgcaaataccaaaagtttgcttgatagcaacctggaggatggaagatacatatctgcccagggtaagaaggtggtggtcattggtggtggagacacaggcacagattgcatcggtacgtctgttaggcatggttgcagcagcattgtaaatctggagcttctcaccaagccaccaagcaagagagcttctgacaacccctggccccagtggcctagagtcttccgagtggactatgggcaccaggaagcatctaccaaatttggaaatgatccaagaacttacgaagtcttaaccaagcgtttcattggtgatgaagatggaaaattgaaggcccttgaggtggtgcgcgtgaagtgggagaaagtagatggaaaattccagttcaaggagattgaaggatcacaagagatcatcgaggcagaccttgtcctccttgccatgggattcctgggccctgaagagaacatcgctgacaaactgggtttagagaaagacaaccgttccaacttcaaagctcaattcggacacttcgggaccagtgtggatggcgtttttgccgctggggattgcaggcgcgggcaatcgctggttgtttgggccatcaccgaagggcgtgaagctgctgctgcagtagataagtacttgtcaagggatgaacaaaatgtcgcaggccttacatag

SEQ ID NO: 27 - DNA, 23 bp, Artificial Sequence
  Primer
cgagcttgaggatttgagttcta

SEQ ID NO: 28 - DNA, 22 bp, Artificial Sequence
  Primer
cacttgctaaactggtataatg

SEQ ID NO: 29 - DNA, 19 bp, Artificial Sequence
  Primer
tcgctgagtctctaggaca

SEQ ID NO: 30 - DNA, 20 bp, Artificial Sequence
  Primer
gttcaatggctggttcagta

SEQ ID NO: 31 - DNA, 24 bp, Artificial Sequence
  Primer
ggatttgaattctgcagagagaaa

SEQ ID NO: 32 - DNA, 22 bp, Artificial Sequence
  Primer
cacttgctaaactggtacaagt

SEQ ID NO: 33 - DNA, 19 bp, Artificial Sequence
  Primer
ctacagagagaagacaggc

SEQ ID NO: 34 - DNA, 25 bp, Artificial Sequence
  Primer
gtacaattgatcctgcacatatact

SEQ ID NO: 35 - DNA, 301 bp, Triticum aestivum
agacaatattgtcattggtaatgtggccctgtatggtgctaccaagggagaagcatacttcaatggaatggcagcagaaaggttttgtgttcgtaattctggtgctcgracagtggttgaaggaattggtgatcatggatgcgagtacatgacagggggtactgtagtcatccttggtaaaacaggaagaaattttgctgctgggatgagtggaggcatcgcttatgtttatgatgttgatgggacattcagtgtccgctgtaacaatgagttggttgatctatatcatgtggaggaagag

SEQ ID NO: 36 - DNA, 196 bp, Triticum aestivum
  variation (112)..(112): the adenine can be deleted
gtctgttaggcatggttgcagcagcattgtaaatctggagcttctcaccaagccaccaagcaagagagcttctgacaacccctggccccaggtaaatgcaaaaaaaaaaaaactggatatttccctgcacatgtgaatttggccattccatttccagcttggaaagttcttaagtcatcattctgtttttgcatgc

SEQ ID NO: 37 - DNA, 206 bp, Triticum aestivum
gggagggaggggagcagccttctcgtatggagcctcttcctgatggttcgatgaatccaaaacgaagtgcaatcaagcaagttgccagtggacgatttggagtttccagctattatctgactaatgcagatgrgctgcagataaaaatggctcaggtacttgacaatgctgtgcattgcgcaatcactggaatgatctcagtttat

SEQ ID NO: 38 - DNA, 179 bp, Triticum aestivum
taacatttgtggtgaatgctaatgtagggagtgatcctttatactcaattgaacgtctccrttctgagaacaatgcagttattttggcttgtggagctacaaaaccaaggtaactaattggaacgaggattttttttttctagtttactccaagagtagcaacaatctcaaaagaactg

SEQ ID NO: 39 - DNA, 518 bp, Triticum aestivum
aggtacacattggaagccctagaaatgttgctgctgccaatggctaaagatggagtggaagctcttggatcaatgggaaatgatgcacccctagcagtgatgtcaaacagagagaagctgacttttgagtacttcaagcagatgtttgcacaagtaacaaaccctccaatcgatccaattagggagaagattgttacatctatggaatgtatgattgggccagaaggagatttgctggaaataaccgaaaagcaatgcaaccgccttgcacttaaaggtcctttggtgtcaatggatgaaatggaatctatcaagaagatgaactaccgtggttggcgcagcaaggtgctcgacataacttatccgaagaattctggaaggaagggcttggaagaaactttggatagaatttgtgctgaagcccgggaagctatacgcragggatacaaaattttagttctttcagacagaggtcagtgacttattcactttatacttgcatcccattgtttgatggt

SEQ ID NO: 40 - DNA, 227 bp, Triticum aestivum
atcataattttgtttgatagtcaattgtttccagtgtaatgtgttgtatgaaggacattcttactttgtcaccttggacataactggaatgtatattaagtttatyatgcaactcaatatcttccaatactgaaatttgttgtattttcttgattcaggattttcttcagatcgtgttgctgtcagttccctcttagcagttggagcagtacatcaacaccttgttg

(19) United States
(12) Patent Application Publication
(10) Pub. No.: US 2013/0047300 A1
(43) Pub. Date: Feb. 21, 2013

(54) Grain Filling of a Plant Through the Modulation of NADH-Glutamate Synthase

(71) Applicants: Jérôme Salse, Valbeleix (FR); Umar Masood Quraishi, Clermont-Ferrand (FR); Caroline Pont, Valbeleix (FR); Florent Murat, Clermont-Ferrand (FR); Jacques Le Gouis, Champeix (FR); Stéphane Lafarge, La Roche Blanche (FR)

(72) Inventors: Jérôme Salse, Valbeleix (FR); Umar Masood Quraishi, Clermont-Ferrand (FR); Caroline Pont, Valbeleix (FR); Florent Murat, Clermont-Ferrand (FR); Jacques Le Gouis, Champeix (FR); Stéphane Lafarge, La Roche Blanche (FR)

(73) Assignee: GENOPLANTE-VALOR, Paris (FR)

(21) Appl. No.: 13/576,610

(22) PCT Filed: Feb. 8, 2011
(86) PCT No.: PCT/IB2011/050534

§ 371 Date: Oct. 19, 2012

(30) Foreign Application Priority Data
Feb. 8, 2010 (EP) 10290058.6

(51) Int. Cl. C12N 15/82; C12N 5/10; A01H 4/00; C12Q 1/68; A01H 5/10; C12N 9/02; C12N 15/53; C12N 15/63; A01H 5/00

(52) U.S. Cl. 800/290; 800/278; 435/320.1; 435/419; 435/412; 435/430; 800/298; 800/320.1; 800/320.3; 435/189; 536/23.2; 435/6.12; 435/6.11

(57) ABSTRACT

The invention relates to a method for increasing the grain filling of a plant, wherein said method comprises overexpressing in said plant a wheat NADH-dependent glutamate synthase, in order to increase the grain weight and/or the grain protein content.

The present invention relates to methods for controlling yield of a plant, preferably a wheat or maize plant, through the modulation of NADH-dependent glutamate synthase (NADH-GoGAT) activity.

High grain yield with adequate protein content is an important goal in crop improvement especially in bread wheat (Triticum aestivum L.) and maize (Zea mays). Unfortunately, it has been shown in various cereals including wheat that these two traits are genetically negatively correlated either in extensive North American farming or in intensive farming in Europe (Simmonds 1995; Oury et al., 2003). This correlation can be broken down by adequate nitrogen (N) supply late in the plant development (Krapp et al., 2005; Laperche et al., 2006). Nitrogen fertilizers are used as an important agronomic tool to improve output quantity as well as quality in all cultivated crops. However, the current agricultural and economic environment concerns impose farmers to constantly optimize the application of nitrogen fertilizers in order to avoid pollution by nitrates and preserve their economic margin.

Therefore, the selection for cereal cultivars that absorb and metabolize nitrogen in the most efficient way for grain or silage production is becoming increasingly important. Such improved crops would make a better use of nitrogen fertilizer supplies as they would produce higher yields with better protein content. This might be achieved, at least in part, through a better understanding of nitrogen metabolism and its regulation, and by identifying target genes to monitor nitrogen uptake by either direct gene transfer or marker-assisted breeding. Either directly for the grain protein content or indirectly for the photosynthetic production in plant, nitrogen uptake is an essential element in crop improvement.

Some genetic variability in nitrogen use efficiency (NUE) and its components, namely nitrogen uptake and nitrogen utilization, has been reported in rice (Borrell et al., 1998) and wheat (Le Gouis et al., 2000). Further, various QTL (Quantitative Trait Loci) analyses for NUE have been performed during the last decades for barley (Kjaer and Jensen, 1995), maize (Agrama et al., 1999; Bertin and Gallais, 2001; Hirel et al., 2001) rice (Obara et al., 2001; Lian et al., 2005), and wheat (An et al. 2006; Laperche et al., 2007; Habash et al., 2007; Fontaine et al., 2009), and for Arabidopsis thaliana (Rauh et al., 2002; Loudet et al., 2003). Major enzyme coding genes have been cloned and shown to drive nitrogen economy in plants (for review Miin and Habash, 2002; Bernard and Habash, 2009).

The glutamine synthetase (GS; E.C.6.3.1.2) is the first key enzyme for nitrogen metabolism, as it catalyses the assimilation of all inorganic nitrogen incorporated into organic compounds, such as proteins and nucleic acids. This reaction is coupled to the formation of glutamate by glutamate synthase (GoGAT) as part of the GS/GoGAT cycle.

In rice, two GoGAT types have been identified: a Ferredoxin (Fd)-dependent GoGAT (E.C. 1.4.7.1) and a NADH-dependent GoGAT (E.C. 1.4.1.14). Fd-GoGAT is known to be involved in photorespiration (Ireland and Lea, 1999). NADH-GoGAT is active in developing organs, such as unexpanded non-green leaves and developing grains (Yamaya et al., 1992).

NADH-GoGAT catalyzes the reductive transfer of amide group of glutamine to 2-oxoglutarate to form two glutamate molecule (Krapp et al., 2005):

2L-glutamate+NAD⁺⇄L-glutamine+2-oxoglutarate+NADH+H⁺.

It is hypothesized that NADH-GoGAT is probably involved in the utilization of remobilized nitrogen, since this protein is located in the specific cell types which are important for solute transport from the phloem and xylem elements (Hayakawa et al., 1994). Yamaya et al. (2002) reported that, in rice, GoGAT enhances the grain filling suggesting that it is one of the potential candidate genes for NUE determinant. However, the authors have shown that in TO transgenic rice plants over-producing NADH-GoGAT, the rate of increase in the NADH-GoGAT protein content in unexpanded non-green leaf blades was inversely correlated with that the one spikelet weight and the panicle weight.

Further, although Ferredoxin-GoGAT plays a critical part in the re-assimilation of ammonium released by glycine decarboxylase during photorespiration, NADH-GoGAT is involved in the assimilation of ammonium from both primary and secondary sources during nitrogen remobilization (Lea and Miin, 2003).

Genes coding for these two key enzymes involved in the NH₄ assimilation (GS and NADH-GoGAT) have been cloned in monocots such as rice (Tabuchi et al., 2007 for both GS and NADH-GoGAT; Cai et al., 2009 for GS), wheat (Caputo et al., 2009 showing a physiological role of GS in the modulation of amino acids export levels in wheat) and maize (Valadier et al., 2008 for both GS and NADH-GoGAT); and eudicots such as Arabidopsis (Ishiyama et al., 2004 for GS; Potel et al. 2009 for NADH-GoGA 7), Brassicaceae (Ochs et al., 1999 for GS) and Medicago (Lima et al., 2006 for GS).

Bread wheat is a hexaploid species with three diploid genomes named A, B and D; each genome consisting of seven pairs of chromosomes. The interactions between these 3 genomes are still unclear. Several putative NADH-GoGAT expressed sequence tags (ESTs), homolog to NADH-GoGAT ESTs in rice, have been found in bread wheat. However, until now, the functional ortholog of rice NADH-GoGAT has not been cloned in bread wheat.

The Inventors have now found, in bread wheat, a NADH-GoGAT gene which plays a major role in driving NUE. This gene is located on chromosome 3B.

The Inventors have also found that the wheat NADH-GoGAT proteins playing a major role in driving NUE show at least 98% identity between them and that such a wheat NADH-GoGAT protein has a percent identity inferior or equal to 95% with the rice NADH-GoGAT, whose the amino acid sequence is available in GENBANK database under accession number GI:115439209 (and herein reproduced as SEQ ID NO: 6).

This finding from the Inventors that NADH-GoGAT protein plays a major role in driving NUE in wheat can also apply to other plants such as maize.

Accordingly, the present invention provides a method for improving the grain filling of a plant, preferably a wheat plant or a maize plant, more preferably a wheat plant, wherein said method comprises overexpressing in said plant a NADH-dependent glutamate synthase (NADH-GoGAT) having at least 95% identity, or by order of increasing preference at least 96%, 97%, 98% or 99% identity, with the polypeptide of sequence SEQ ID NO: 1.

Unless otherwise specified, the percents of identity between two sequences which are mentioned herein are calculated from an alignment of the two sequences over their whole length.

The term “overexpressing” a NADH-dependent glutamate synthase (NADH-GoGAT) in a plant, herein refers to artificially increasing the quantity of said NADH-GoGAT produced in said plant compared to a reference (control) plant.

The term “plant” includes any monocot or dicot plant producing edible seeds. Preferably, said plant is a wheat plant or a maize plant, more preferably a wheat plant.

The terms “wheat plant” and “wheat plant cell” as used herein, include any plant or plant cell of the genus Triticum, preferably of the species Triticum aestivum L. (bread wheat).

The terms “maize plant” and “maize plant cell” as used herein, include any plant or plant cell of the genus Zea, preferably of the species Zea mays, more preferably of the subspecies Zea mays mays.

According to a preferred embodiment of the invention the grain filling is improved by increasing the grain weight and/or the grain protein content.

Advantageously, the improvement of the grain filling involves an improvement of the grain yield, either in limited or non-limited nitrogen supply condition.

According to another preferred embodiment of the invention said NADH-GoGAT has the amino acid sequence SEQ ID NO: 22, which corresponds to the NADH-GoGAT amino acid sequence of the bread wheat cultivar Chinese Spring. This sequence has 99.6% identity with the sequence SEQ ID NO: 1.

According to another preferred embodiment of the invention said NADH-GoGAT is encoded by a nucleotide sequence selected from the group consisting of SEQ ID NO: 2, SEQ ID NO: 3, SEQ ID NO: 4, SEQ ID NO: 23, SEQ ID NO: 24 and SEQ ID NO: 25, more preferably by a nucleotide sequence selected from the group consisting of SEQ ID NO: 23, SEQ ID NO: 24 and SEQ ID NO: 25, which correspond respectively to the genomic DNA sequences (allele) encoding the NADH-GoGAT protein of the bread wheat cultivars Chinese Spring, Arche and Récital.

According to another preferred embodiment of the invention said NADH-GoGAT is encoded by the nucleotide sequence SEQ ID NO: 5 or SEQ ID NO: 26, preferably by the nucleotide sequence SEQ ID NO: 26, which corresponds to the coding DNA sequence (CDS) of Chinese Spring NADH-GoGAT gene.

A preferred method for overexpressing a NADH-GoGAT comprises introducing into the genome of said plant a DNA construct comprising a nucleotide sequence encoding said NADH-GoGAT, placed under control of a promoter.

The instant invention also provides means for carrying out said overexpression.

This includes, in particular, recombinant DNA constructs for expressing a NADH-GoGAT in a host-cell (e.g., plant cell), or a host organism, in particular a wheat or maize plant cell or a wheat or maize plant. These DNA constructs can be obtained and introduced in said host cell or organism by the well-known techniques of recombinant DNA and genetic engineering.

Recombinant DNA constructs of the invention include in particular expression cassettes, comprising a polynucleotide encoding a NADH-GoGAT as defined above, under control of a heterologous promoter functional in plant cell.

The expression cassette of the invention may comprise a polynucleotide encoding at least two identical or different NADH-GoGAT as defined above.

The heterologous promoter of the invention is any promoter functional in a plant cell, i.e., capable of directing transcription of a polynucleotide encoding a NADH-GoGAT as defined above, in said plant cell. The choice of the more appropriate promoter may depend in particular on the organ(s) or tissue(s) targeted for expression, and on the type of expression (i.e. constitutive or inducible) that one wishes to obtain.

A large choice of promoters suitable for expression of heterologous genes in plants is available in the art. They can be obtained for instance from plants, plant viruses, or bacteria such as Agrobacterium. They include constitutive promoters, i.e. promoters which are active in most tissues and cells and under most environmental conditions, tissue or cell specific promoters which are active only or mainly in certain tissues or certain cell types, and inducible promoters that are activated by physical or chemical stimuli.

Non-limitative examples of constitutive promoters that are commonly used are the cauliflower mosaic virus (CaMV) 35S promoter, the nopaline synthase (Nos) promoter, the Cassava vein Mosaic Virus (CsVMV) promoter (Verdaguer et al., 1996), the rice actin promoter followed by the rice actin intron (RAP-RAI) contained in the plasmid pAct1-F4 (McElroy et al., 1991).

Non-limitative examples of organ or tissue specific promoters that can be used in the present invention include for instance High Molecular Weight (HMW) promoter which is kernel specific (Thomas and Flavell, 1990), or the leaf specific promoters as pPEPc promoter (Jeanneau et al., 2002), or the Rubisco small subunit promoter (rbcS) (Katayama et al., 2000) which is specific of the bundle-sheath, or the root specific promoter PRO110 from rice (International Application WO 2004/070039).

Inducible promoters include for instance drought stress responsive promoters, such as the rd29A promoter which comprises a dehydration-responsive element (Kasuga et al., 1999; Narusaka et al., 2003), or the senescence specific SAG12 promoter (Noh and Amasino, 1999).

The expression cassettes generally also include a transcriptional terminator, such as the 35S transcriptional terminator or Nos terminator (Depicker et al., 1982). They may also include other regulatory sequences, such as transcription enhancer sequences.

Recombinant DNA constructs of the invention also include recombinant vectors containing an expression cassette comprising a polynucleotide encoding a NADH-GoGAT as defined above, under transcriptional control of a suitable promoter. Said expression cassette may be a recombinant expression cassette of the invention, or a cassette wherein the polynucleotide encoding a NADH-GoGAT is under control of its endogenous promoter.

A recombinant vector of the invention may include at least two polynucleotides encoding two identical or different NADH-GoGAT as defined above.

Recombinant vectors of the invention may also include other sequences of interest, such as, for instance, one or more marker genes, which allow for selection of transformed hosts.

Advantageously, the selectable marker gene is comprised between two Ds (Dissociation) elements (i.e., transposons) in order for its removal at a later stage by interacting with the Ac (Activator) transposase. This elimination system is known from one skilled in the art. By way of example, it has been described in Goldsbrough et al. (1993).

The selection of suitable vectors and the methods for inserting DNA constructs therein are well known to persons of ordinary skill in the art. The choice of the vector depends on the intended host and on the intended method of transformation of said host.

A variety of techniques for genetic transformation of plant cells (e.g., wheat or maize plant cells), or plants (e.g., wheat or maize plants) are available in the art. By way of non-limitative examples, one can mention methods of direct transfer of genes such as direct micro-injection into plant embryoids, vacuum infiltration (Bechtold et al. 1993) or electroporation (Chupeau et al., 1989), or the bombardment by gun of particules covered with the plasmidic DNA of interest (Fromm et al., 1990; Finer et al., 1992). Agrobacterium mediated transformation methods may also be used such as Agrobacterium tumefaciens, in particular according to the method described in the article by An et al. (1986), or Agrobacterium rhizogenes, in particular according to the method described in the article by Guerche et al., (1987). According to a particular embodiment, it is possible to use the method described by Ishida et al. (1996) for the transformation of maize. According to another embodiment, the wheat is transformed according to the method described in International Application WO 00/63398.

The invention also comprises host cells containing a recombinant DNA construct of the invention. These host cells can be prokaryotic cells or eukaryotic cells, in particular plant cells, and preferably wheat or maize plant cells.

The invention also provides a method for producing a transgenic plant, preferably a transgenic wheat or maize plant, having an improved grain filling. Said method comprises transforming a plant cell by a DNA construct of the invention and regenerating from said plant cell a transgenic plant overexpressing a NADH-GoGAT as defined above.

According to a preferred embodiment of the method of the invention, it comprises transforming a plant cell by a recombinant vector of the invention comprising a polynucleotide encoding a NADH-GoGAT as defined above, and regenerating from said plant cell a transgenic plant overexpressing a NADH-GoGAT as defined above.

The invention also comprises plants, preferably wheat or maize plants, genetically transformed by a recombinant DNA construct of the invention, and overexpressing a NADH-GoGAT as defined above. In said transgenic plants a DNA construct of the invention is comprised in a transgene stably integrated in the plant genome, so that it is passed onto successive plant generations. Thus the transgenic plants of the invention include not only the plants resulting from the initial transgenesis, but also their descendants, as far as they contain a recombinant DNA construct of the invention. The overexpression of a NADH-GoGAT as defined above in said plants provides them an improved grain filling, when compared with a plant devoid of said transgene(s).

The invention also comprises a transgenic plant, preferably a transgenic wheat or maize plant, obtainable by a method of the invention, overexpressing a NADG-GoGAT as defined above, said plant containing a recombinant expression cassette of the invention.

The invention further comprises a transgenic plant, preferably a transgenic wheat or maize plant, or an isolated organ or tissue thereof comprising, stably integrated in its genome, a recombinant expression cassette comprising a polynucleotide encoding a NADH-GoGAT as defined above.

Accordingly, the invention also encompasses isolated organs or tissues of said transgenic plant (such as seeds, leafs, flowers, roots, stems, ears) containing a recombinant expression cassette of the invention.

The present invention also provides an isolated wheat NADH-dependent glutamate synthase protein having at least 95% identity with the polypeptide of sequence SEQ ID NO: 1. Preferably, said NADH-dependent glutamate synthase protein has the amino acid sequence SEQ ID NO: 22.

The present invention also provides an isolated polynucleotide chosen from the group consisting of:

a) a polynucleotide encoding a wheat NADH-GoGAT involved in Nitrogen Use Efficiency, which polypeptide has at least 95%, or by order of increasing preference at least 96%, 97%, 98% or 99% identity, with the polypeptide of sequence SEQ ID NO: 1;

b) a polynucleotide complementary to the polynucleotide a);

c) a polynucleotide capable of hybridizing selectively, under stringent conditions, with the polynucleotide a) or the polynucleotide b).

According to a preferred embodiment, the polynucleotide encoding a wheat NADH-GoGAT is selected from the group consisting of sequences SEQ ID NO: 2, 3, 4, 5, 23, 24, 25 and 26, preferably selected from the group consisting of sequences SEQ ID NO: 23, 24, 25 and 26.

Stringent hybridization conditions, for a given nucleotide, can be identified by those skilled in the art according to the size and the base composition of the polynucleotide concerned, and also according to the composition of the hybridization mixture (in particular pH and ionic strength). Generally, stringent conditions, for a polynucleotide of given size and given sequence, are obtained by carrying out procedures at a temperature approximately 5° C. to 10° C. below the melting temperature (Tm) of the hybrid formed, in the same reaction mixture, by this polynucleotide and the polynucleotide complementary thereto.

A “polynucleotide capable of hybridizing selectively with a polynucleotide a) or b) in accordance with the invention” is here defined as any polynucleotide which, when it is hybridized under stringent conditions with a wheat nucleic acid library (in particular a genomic DNA or cDNA library), produces a detectable hybridization signal (i.e. at least twice as great, preferably at least five times as great, as the background noise) with said polynucleotide, but produces no detectable signal with other sequences of said library, and in particular with sequences encoding other proteins of the GoGAT family.

A subject of the present invention is also polynucleotide probes or amplification primers obtained from polynucleotides a) or b) in accordance with the invention or fragments thereof.

The present invention also encompasses any polynucleotide encoding a wheat NADH-GoGAT involved in Nitrogen Use Efficiency (NUE) and which can be obtained from a plant genomic DNA or cDNA library by screening said library with probes or primers in accordance with the invention. This includes in particular other alleles of the wheat NADH-GoGAT gene, and in particular other alleles capable of conferring an improved NUE and/or grain filling.

By way of example, one can also use at least one of the following pairs of primers:
TTAGTGGCAAATGGGCTTCG (SEQ ID NO: 7) and CGCCACAGCAACATCTCTACC (SEQ ID NO: 8);
CAGCTGCAGAGATTCGTCCTG (SEQ ID NO: 9) and TGTTATCCAAAGCCATGTCAAGG (SEQ ID NO: 10);
TGGAATGGCAGCAGAAAGGT (SEQ ID NO: 11) and TCGCATCCATGATCACCAATT (SEQ ID NO: 12);
GCACCATTTCTGTACACTCGTTG (SEQ ID NO: 13) and ATCTTCCCATCAGTCTGCAAGC (SEQ ID NO: 14);
TTCAAGAGCTTAACAAGGCGTG (SEQ ID NO: 15) and CACTTGCAGGTTCAACCTCATC (SEQ ID NO: 16);
TGGGAAATGATGCACCCCTA (SEQ ID NO: 17) and CTTGTGCAAACATCTGCTTGAAG (SEQ ID NO: 18);
AATTCTGGAAGGAAGGGCTTG (SEQ ID NO: 19) and TTTGTATCCCTCGCGTATAGCTT (SEQ ID NO: 20);
CGAGCTTGAGGATTTGAGTTCTA (SEQ ID NO: 27) and CACTTGCTAAACTGGTATAATG (SEQ ID NO: 28), useful to amplify a fragment of a wheat NADH-GoGAT gene on chromosome 3A;
TCGCTGAGTCTCTAGGACA (SEQ ID NO: 29) and GTTCAATGGCTGGTTCAGTA (SEQ ID NO: 30), useful to amplify a fragment of a wheat NADH-GoGAT gene on chromosome 3B;
GGATTTGAATTCTGCAGAGAGAAA (SEQ ID NO: 31) and CACTTGCTAAACTGGTACAAGT (SEQ ID NO: 32), useful to amplify a fragment of a wheat NADH-GoGAT gene on chromosome 3B;
CTACAGAGAGAAGACAGGC (SEQ ID NO: 33) and GTACAATTGATCCTGCACATATACT (SEQ ID NO: 34), useful to amplify a fragment of a wheat NADH-GoGAT gene on chromosome 3D;

preferably at least one of the following pairs of primers selected from the group consisting of SEQ ID NO: 27 and 28, SEQ ID NO: 29 and 30, SEQ ID NO: 31 and 32, and SEQ ID NO: 33 and 34.

The invention also provides means for identifying and selecting wheat plants which have an improved grain filling compared to a reference wheat plant.

The invention thus provides a method for identifying an allele of a wheat NADH-GoGAT gene associated with a given phenotype of grain filling, wherein said method comprises isolating a nucleic acid fragment comprising said NADH-GoGAT gene or a portion thereof from at least one wheat plant expressing said phenotype, and sequencing said fragment.

The invention further provides a method for identifying polymorphisms associated with grain filling, in a NADH-GoGAT gene, wherein said method comprises identifying, as described above, at least two different alleles of said NADH-GoGAT gene associated with different phenotypes of grain filling, and comparing the sequences of said alleles.

Based on the NADH-GoGAT allele sequences characterised in wheat genotypes, the Inventors have identified 6 DNA sequence variations (5 Single Nucleotide Polymorphisms (SNPs) and 1 Insertion/Deletion (InDel)), represented by the sequences SEQ ID NO: 35, 36, 37, 38, 39 and 40, that can be used in Marker Assisted Selection (MAS) breeding programs for improving the grain filling of a wheat plant (NUE improvement for instance).

The Inventors have also identified, in Chinese Spring, Arche and Récital genotypes, 23 other DNA sequence variations (18 Single Nucleotide Polymorphisms (SNPs) and 5 Insertion/Deletion (InDels)) shown in Table 1 below, that can be used in Marker Assisted Selection (MAS) breeding programs for improving the grain filling of a wheat plant (NUE improvement for instance).

TABLE 1
Detailed information regarding 18 SNP and 5 InDels identified
between Chinese Spring, Récital and Arche
genotypes. SNP and InDels coordinates are based on the
Chinese Spring (CS) allele (SEQ ID NO: 2).

| Base Coordinate (CS allele) | Chinese Spring | Arche | Récital |
|---|---|---|---|
| #2545 | A | A | G |
| #2663 | A | G | A |
| #2737 | G | G | A |
| #2871 | T | T | C |
| #2892 | G | G | T |
| #3010 | G | G | A |
| #3039 | A | A | G |
| #3512 | G | A | G |
| #4752 | G | G | C |
| #5426 | C | C | T |
| #5452 | x | x | TA |
| #5509 | G | G | A |
| #5681 | G | A | A |
| #6420 | x | G | G |
| #6916 | G | x | G |
| #8253 | A | G | G |
| #8882 | G | x | A |
| #8943 | A | G | G |
| #9404 | A | A | x |
| #9489 | T | A | A |
| #9541 | A | G | G |
| #9566 | T | G | G |
| #9592 | G | x | x |

A, C, G and T represent the 4 nucleotide bases, repectively adenine, cytosine, guanine and thymine. “x” represents a deletion.

Once a polymorphism has been identified, reagents and kits allowing the routine detection of said polymorphism can be designed. Commonly used reagents are nucleic acid probes, or restriction enzymes, or PCR primers, or combinations thereof. The choice of a reagent or of a combination of reagents depends of the nature of the polymorphism.

Preferred kits and reagents are those comprising a set of primers allowing specific PCR amplification of a DNA segment spanning the polymorphic locus. For microsatellites and insertion/deletion polymorphisms, PCR primers may be sufficient, since the allelic forms of the polymorphism may be differentiated by the size of the amplification product. In the case of single nucleotide polymorphisms (SNP), one will generally also use a restriction enzyme, which allows the differentiation of allelic forms by the presence or size of restriction fragments.

For these purposes, it is possible to use a nucleic acid encoding a NADH-GoGAT as defined above, or a fragment thereof, as a probe or a target for amplification, for selecting wheat plants naturally overexpressing a NADH-GoGAT as defined above, and therefore exhibiting an improved grain filling. Preferably, the amplified fragment has a length of about 500 pb, more preferably, of about 500 to 1000 pb.

The invention also provides a method for identifying in a wheat plant (a) genetic marker(s) associated with an improved grain filling, said method comprising genotyping said wheat plant and identifying one or more of the following alleles encoding an NADH-GoGAT as defined above:
an allele comprising the sequence SEQ ID NO: 35 wherein the nucleotide at position 109 of said sequence is guanine (corresponding to the favourable allele for improving the grain filing in cultivar Arche);
an allele comprising the sequence SEQ ID NO: 36, wherein a nucleotide adenine is present at position 112 of said sequence (corresponding to the favourable allele for improving the grain filing in cultivar Arche);
an allele comprising the sequence SEQ ID NO: 37 wherein the nucleotide at position 133 of said sequence is adenine (corresponding to the favourable allele for improving the grain filing in cultivar Arche);
an allele comprising the sequence SEQ ID NO: 38 wherein the nucleotide at position 61 of said sequence is guanine (corresponding to the favourable allele for improving the grain filing in cultivar Arche);
an allele comprising the sequence SEQ ID NO: 39 wherein the nucleotide at position 439 of said sequence is guanine (corresponding to the favourable allele for improving the grain filing in cultivar Arche);
an allele comprising the sequence SEQ ID NO: 40 wherein the nucleotide at position 106 of said sequence is thymine.

Many techniques are known by the person skilled in art to identify a specific allele. By way of example, said allele can be identified by sequencing or by hybridization with a nucleotide sequence complementary to the sequences SEQ ID NO: 35-40 respectively. Said allele can be amplified using a pair of primers according to the present invention as defined above.

The invention further provides a method for selecting a wheat plant having an improved grain filling, wherein said method comprises identifying in wheat plants to be tested (a) genetic marker(s) associated with an improved grain filling by the method defined above, and selecting a plant containing said genetic marker(s).

The Inventors also disclose a method for inhibiting in a plant, preferably a wheat or maize plant, a NADH-dependent glutamate synthase (NADH-GoGAT) having at least 95% identity, or by order of increasing preference at least 96%, 97%, 98% or 99% identity, with the polypeptide of sequence SEQ ID NO: 1 or SEQ ID NO: 22 as defined above.

The inhibition of a NADH-GoGAT protein can be obtained either by abolishing, blocking or decreasing its function (i.e. catalyzing the reductive transfer of amide group of glutamine to 2-oxoglutarate to form two glutamate molecule), or advantageously, by preventing or down-regulating the expression of its gene.

By way of example, inhibition of said NADH-GoGAT protein can be obtained by mutagenesis of the corresponding gene or of its promoter, and selection of the mutants having partially or totally lost the NADH-GoGAT protein activity. For instance, a mutation within the coding sequence can induce, depending on the nature of the mutation, the expression of an inactive protein, or of a protein with impaired activity; in the same way, a mutation within the promoter sequence can induce a lack of expression of said NADH-GoGAT protein, or decrease thereof.

Mutagenesis can be performed for instance by targeted deletion of the NADH-GoGAT coding sequence or promoter, or of a portion thereof, or by targeted insertion of an exogenous sequence within said coding sequence or said promoter. It can also be performed by random chemical or physical mutagenesis, followed by screening of the mutants within the NADH-GoGAT gene. Methods for high throughput mutagenesis and screening are available in the art. By way of example, one can mention TILLING (Targeting Induced Local Lesions IN Genomes, described by McCallum et al., 2000).

Advantageously, the inhibition of said NADH-GoGAT protein is obtained by silencing of the corresponding gene. Methods for gene silencing in plants are known in themselves in the art. For instance, one can mention by antisense inhibition or co-suppression, as described by way of example in U.S. Pat. Nos. 5,190,065 and 5,283,323. It is also possible to use ribozymes targeting the mRNA of said NADH-GoGAT protein.

Preferred methods are those wherein post transcriptional gene silencing is induced by means of RNA interference (RNAi) targeting the NADH-GoGAT gene to be silenced. Various methods and DNA constructs for delivery of RNAi are available in the art (for review, Watson et al., 2005). Typically, DNA constructs for delivering RNAi in a plant include at least a fragment of 300 bp or more (generally 300-800 bp, although shorter sequences may sometime induce efficient silencing) of the cDNA of the target gene, under transcriptional control of a promoter active in said plant. Currently, the more widely used DNA constructs are those that encode hairpin RNA (hpRNA). In these constructs, the fragment of the target gene is inversely repeated, with generally a spacer region between the repeats.

The Inventors further disclose chimeric DNA constructs for silencing a NADH-GoGAT gene.

Such a chimeric DNA construct comprises:
a promoter functional in a plant cell;
a DNA sequence of 200 to 1000 bp, preferably of 300 to 900 bp, consisting of a fragment of a cDNA encoding a NADH-GoGAT protein or of its complementary, or having at least 95% identity, and by order of increasing preference, at least 96%, 97%, 98% or 99% identity with said fragment, said DNA sequence being placed under transcriptional control of said promoter.

According to a preferred embodiment, said chimeric DNA construct comprises:
a first DNA sequence of 200 to 1000 bp, preferably of 300 to 900 bp, consisting of a fragment of a cDNA encoding a NADH-GoGAT protein, or having at least 95% identity, and by order of increasing preference, at least 96%, 97%, 98% or 99% identity with said fragment;
a second DNA sequence that is the complementary of said first DNA, said first and second sequences being in opposite orientations;
a spacer sequence separating said first and second sequence, such that these first and second DNA sequences are capable, when transcribed, of forming a single double-stranded RNA molecule.

The spacer can be a random fragment of DNA. However, preferably, one will use an intron which is spliceable by the target plant cell. Its size is generally 400 to 2000 nucleotides in length.

A large choice of promoters suitable for expression of heterologous genes in plants is available in the art. They can be chosen among those disclosed above.

DNA constructs for silencing a NADH-GoGAT gene as defined above generally also include a transcriptional terminator (for instance the 35S transcriptional terminator, or the nopaline synthase (Nos) transcriptional terminator).

These DNA constructs for silencing a NADH-GoGAT gene as defined above can be obtained and introduced in a host cell or organism by the well-known techniques of recombinant DNA and genetic engineering, such as those described above.

The Inventors further disclose plant cells (preferably wheat or maize plant cells) or plants (preferably wheat or maize plants) genetically modified by a DNA construct for silencing a NADH-GoGAT gene as defined above. The polynucleotide may be transiently expressed; it can also be incorporated in a stable extrachromosomal replicon, or integrated in the chromosome.

In particular the Inventors disclose a transgenic plant, preferably a transgenic wheat or maize plant, containing a transgene comprising a DNA construct for silencing a NADH-GoGAT gene as defined above.

Foregoing and other objects and advantages of the invention will become more apparent from the following detailed description and accompanying drawing. It is to be understood however that this foregoing detailed description is exemplary only and is not restrictive of the invention.

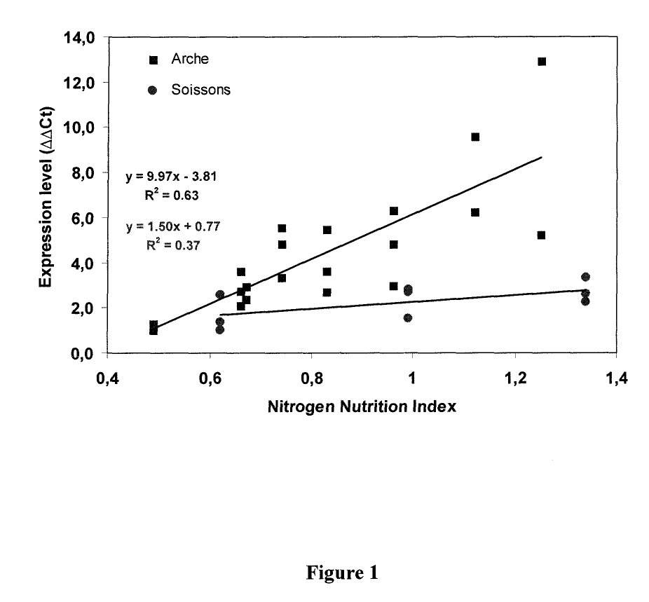

FIG. 1 represents the linear regression observed between the GoGAT gene expression (expressed as ΔΔCT) and the NNI status of the Arche (square) and Soissons (round) wheat genotypes for 29 leaf samples collected after flowering (respectively at Z75 and Z65).

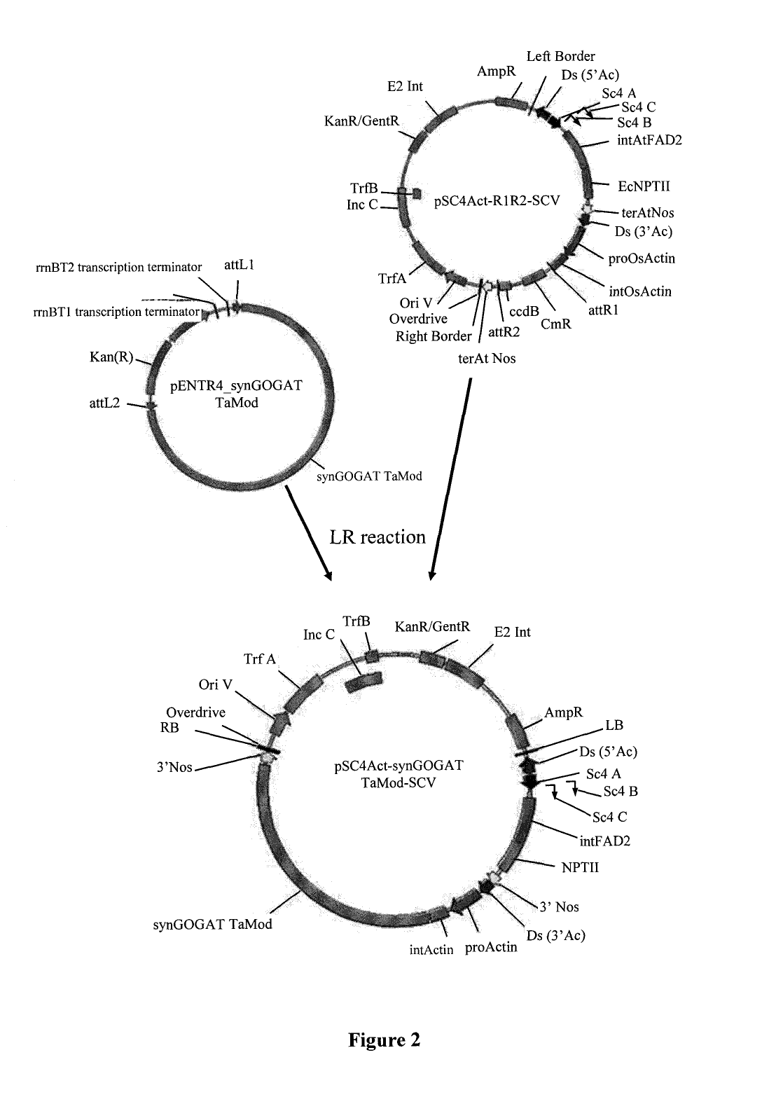

FIG. 2 shows the cloning strategy for pSC4Act-synGOGAT TaMod-SCV.

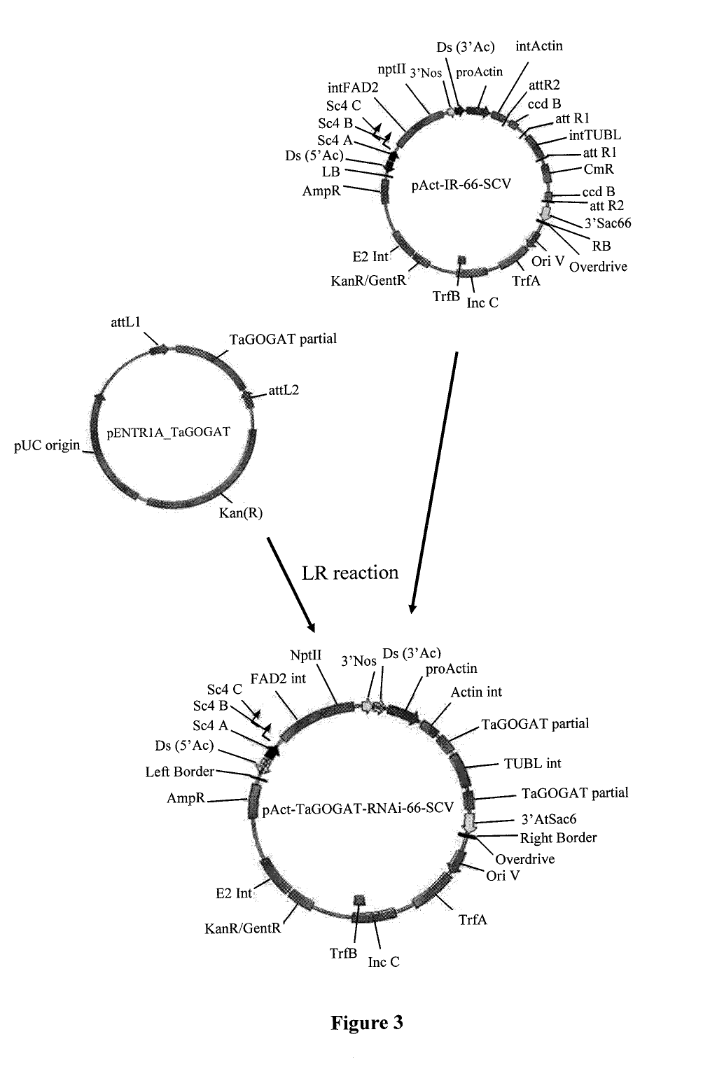

FIG. 3 shows the cloning strategy for pAct-TaGOGAT-RNAi-66-SCV.

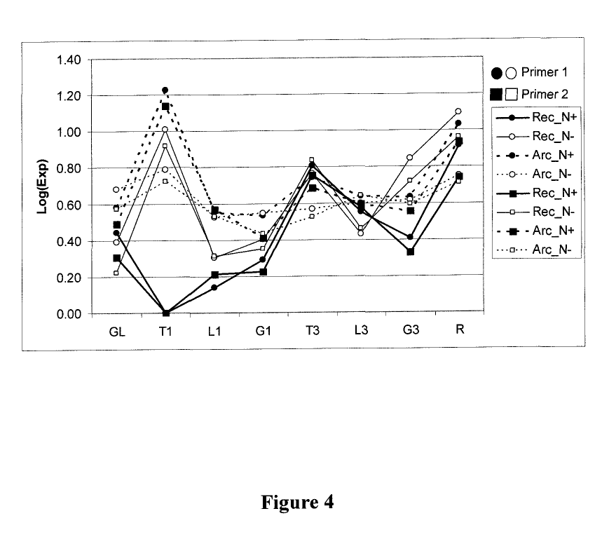

FIG. 4 shows the difference in NADH-GoGAT expression between Arche and Recital wheat genotypes under different N supply levels.

### EXAMPLE 1

### Experimental Validation of the NADH-GoGAT Gene in Nitrogen Use Efficiency (NUE) in Wheat

1) Materials & Methods

Wheat leaf samples were collected on 2 trials (La Minière and Boigneville stations—Arvalis Institut du Végétal; France): one in field for cultivar Arche and the other in green house for cultivar Soissons. Different nitrogen treatments were applied to lead to samples with a range of Nitrogen Nutrional Index (NNI) from 0.49 to 1.34 after flowering. During wheat culture, sampling has been done at 2 stages corresponding to the Zadoks scale: Z65 (Soissons) and Z75 (Arche).

Total RNAs were extracted from all the samples with the SV96 Total RNA Isolation System (Promega) according to the manufacturer instructions. RNA integrity was verified on the Agilent Bioanalyzer and presence of potential genomic DNA was checked by qPCR on RNA. In the absence of genomic DNA no amplification is expected from RNA.

For each sample 2 μg of total RNA were submitted to the reverse transcription using the High capacity reverse transcription kit (Applied Biosystems) and random primers in 100 μl. RT reaction was then 1/10^{th} diluted and 2 μl of cDNA used for the amplification. Each RNA sample was submitted to 2 independent RT reactions for technical reproducibility evaluation.

Quantitative PCR was performed on an ABI7900 machine (Applied Biosystems), using Applied Biosystems reagents. The PCR reactions consisted of a hot-start Taq Polymerase activation step of 95° C. for 5 minutes, followed by 2 steps amplification cycles (denaturation 95° C., 30 sec, annealing/elongation 60° C., 1 min). Expression levels of mRNA for NADH-GoGAT gene were calculated using the Ct estimated by the SDS software (Applied Biosystems) and normalized across samples using 4 control genes. Normalized and Relative expression was then considered as the ΔC and ΔΔCt respectively, between NADH-GoGAT gene and the average of controls.

2) Results

In order to validate the role of the NADH-GoGAT gene in NUE, an experiment on two bread wheat genotypes, i.e. Arche and Soissons, was conducted. Twenty nine leaf samples for Arche and nine for Soissons were collected after flowering (respectively at Z75 and Z65). The N nutrition index (NNI) value was calculated (ranking from 0.49 to 1.34) for each sample. Moreover, for the same samples, RNA was extracted and the expression pattern of GoGAT was analysed through qPCR (ranking from 0 to 14 ΔΔCT) using sequence primers based on the 3B contig sequence (forward: AATTCTGGAAGGAAGGGCTTG; SEQ ID NO: 19; reverse: TTTGTATCCCTCGCGTATAGCTT; SEQ ID NO: 20). The results are shown in Table 2 here-after.

TABLE 2
GoGAT gene expression analysed through qPCR (expressed
as ΔCT and ΔΔCT) and Nitrogen Nutrition Index (NNI
value) for 29 leaf samples on Arche and Soissons genotypes.
The ΔΔCT value of the Z75N1F2 and Z65_F1_T1
samples was set to 1.

| | | | |
|---|---|---|---|
| Sample name | NNI value | ΔCT value | ΔΔCT value |

Arche

| | | | |
|---|---|---|---|
| Z75N1F1 | 0.49 | 8.99 | 1.30 |
| Z75N1F2 | 0.49 | 9.37 | 1.00 |
| Z75N2F1 | 0.66 | 7.91 | 2.75 |
| Z75N2F2 | 0.66 | 8.32 | 2.07 |
| Z75N2F3 | 0.66 | 7.52 | 3.60 |
| Z75N3F1 | 0.67 | 7.82 | 2.94 |
| Z75N3F2 | 0.67 | 8.14 | 2.35 |
| Z75N4F1 | 0.83 | 7.93 | 2.71 |
| Z75N4F2 | 0.83 | 7.51 | 3.63 |
| Z75N4F3 | 0.83 | 6.92 | 5.46 |
| Z75N5F1 | 0.74 | 7.62 | 3.36 |
| Z75N5F2 | 0.74 | 6.90 | 5.56 |
| Z75N5F3 | 0.74 | 7.10 | 4.82 |
| Z75N6F1 | 0.96 | 7.11 | 4.81 |
| Z75N6F2 | 0.96 | 6.71 | 6.31 |
| Z75N6F3 | 0.96 | 7.80 | 2.96 |
| Z75N7F1 | 1.12 | 6.73 | 6.26 |
| Z75N7F2 | 1.12 | 6.11 | 9.55 |
| Z75N8F1 | 1.25 | 6.99 | 5.23 |
| Z75N8F2 | 1.25 | 5.68 | 12.93 |

Soissons

| | | | |
|---|---|---|---|
| Z65_F1_T1 | 0.62 | 8.1 | 1.00 |
| Z65_F1_T2 | 0.99 | 7.5 | 1.54 |
| Z65_F1_T3 | 1.34 | 6.7 | 2.63 |
| Z65_F2_T1 | 0.62 | 6.7 | 2.59 |
| Z65_F2_T2 | 0.99 | 6.6 | 2.80 |
| Z65_F2_T3 | 1.34 | 6.4 | 3.34 |
| Z65_F3_T1 | 0.62 | 7.6 | 1.38 |
| Z65_F3_T2 | 0.99 | 6.7 | 2.68 |
| Z65_F3_T3 | 1.34 | 6.9 | 2.25 |

A significant correlation of R²=63% and 37% was found between the expression (ΔΔCT values) of the NADH-GoGAT gene and the NNI score of the 29 leaves samples for both the Arche and Soissons genotypes, respectively. These results confirm that the NADH-GoGAT gene is the major candidate gene driving NUE on chromosome 3B (FIG. 1).

### EXAMPLE 2

### Construction of Transgenic Wheat Plants Overexpressing a Wheat NADH-GoGAT

1) Wheat Transformation Constructs for NADH-GoGAT Over-Expression

The NcoI-XbaI synthetic fragment of the wheat NADH-GOGAT is cloned in the pUC57 vector (GenBank accession number: Y14837 (GI:2440162)), leading to the pUC57_synGOGAT TaMod vector. The NcoI-XbaI GOGAT fragment from pUC57_synGOGAT TaMod is then introduced in the pENTR4 vector (Invitrogen) linearised with NcoI-XbaI, to create the pENTR4_synGOGAT TaMod.

An LR clonase reaction between the pENTR4_synGOGAT TaMod and the pSC4Act-R1R2-SCV, allows the creation of pSC4Act-synGOGAT TaMod-SCV (FIG. 2). pSC4Act-R1R2-SCV is a vector using the Gateway approach to introduce genes to be expressed under the control of the rice Actin gene promoter (McElroy et al., 1990). pSC4Act-R1R2-SCV is obtained after introduction of the proActin-R1R2-terNOS cassette into the binary vector pSCV1 (Firek et al., 1993). The binary plasmid pSC4Act-synGOGAT TaMod-SCV, is then introduced in the A. tumefaciens hypervirulent strain EHA105, and used for transformation experiments.

2) Wheat Transformation Protocol

The method is essentially similar to the one described in International Application WO 00/63398. Wheat tillers, approximately 14 days post-anthesis (embryos approximately 1 mm in length), are harvested from glasshouse grown plants to include 50 cm tiller stem (22/15° C. day/night temperature, with supplemented light to give a 16 hour day). All leaves are then removed except the flag leaf and the flag leaf is cleaned to remove contaminating fungal spores. The glumes of each spikelet and the lemma from the first two florets are then carefully removed to expose the immature seeds. Only these two seeds in each spikelet are generally uncovered. This procedure is carried out along the entire length of the inflorescence. The ears are then sprayed with 70% IMS as a brief surface sterilization.

Agrobacterium tumefaciens strains containing the vector for transformation are grown on solidified YEP media with 20 mg/l kanamycin sulphate at 27° C. for 2 days. Bacteria are then collected and re-suspended in TSIM1 (MS media with 100 mg/l myo-inositol, 10 g/l glucose, 50 mg/l MES buffer pH5.5) containing 400 μM acetosyringone to an optical density of 2.4 at 650 nm.

Agrobacterium suspension (1 μl) is inoculated into the immature seed approximately at the position of the scutellum:endosperm interface, using a 10 μl Hamilton, so that all exposed seed are inoculated. Tillers are then placed in water, covered with a translucent plastic bag to prevent seed dehydration, and placed in a lit incubator for 3 days at 23° C., 16 hr day, 45μEm-2s-1 PAR.

After 3 days of co-cultivation, inoculated immature seeds are removed and surface sterilized (30 seconds in 70% ethanol, then 20 minutes in 20% Domestos, followed by thorough washing in sterile distilled water). Immature embryos are aseptically isolated and placed on W4 medium (MS with 20 g/l sucrose, 2 mg/l 2,4-D, 500 mg/l Glutamine, 100 mg/l Casein hydrolysate, 150 mg/l Timentin, pH5.8, solidified with 6 g/l agarose) and with the scutellum uppermost. Cultures are placed at 25° C. in the light (16 hour day). After 12 days cultivation on W4, embryogenic calli are transferred to W425G media (W4 with 25 mg/l Geneticin (G418)). Calli are maintained on this media for 2 weeks and then each callus is divided into 2 mm pieces and re-plated onto W425G.

After a further 2 week culture, all tissues are assessed for development of embryogenic callus: any callus showing signs of continued development after 4 weeks on selection is transferred to regeneration media MRM 2K 25G (MS with 20 g/l sucrose, 2 mg/l Kinetin, 25 mg/l Geneticin (G418), pH5.8, solidified with 6 g/l agarose). Shoots are regenerated within 4 weeks on this media and then transferred to MS20 (MS with 20 g/l sucrose, pH5.8, solidified with 7 g/l agar) for shoot elongation and rooting.

The presence of the T-DNA, and the number of copies are quantified by quantitative PCR (qPCR).

### EXAMPLE 3

### Association Studies

The aim of association studies is to identify loci contributing to quantitative traits, based on statistical association between genotypes and phenotypes using a large germplasm collection (panel) without knowledge on pedigree. At the opposite of linkage mapping, association studies can be performed using a selection of cultivars without the need for crossing and screening offspring. In this way, it can be looked at a maximum of genotypic variability (depending on panel selection) in a single study. Thus, using this technique, it is possible to identify favorable alleles of the NADH-GoGAT gene linked to phenotypic data, with a high resolution.

After identification of QTL's NADH-GoGAT gene, a SNP discovery has been carried out by sequencing this gene in several genotypes. Several SNPs have been identified and have been genotyped in a panel of 200 varieties using the SNP/InDel Genotyping Service of KBioscience (Kaspar Technology; http://www.kbioscience.co.uk). Genotyping data have been used for association studies using both General Linear Model (GLM) and Mixed linear model (MLM), and also using structure and Kinship matrix information.

One SNP (namely SNP_{—}3927; shown in SEQ ID NO: 40) located at position 3927 in the coding sequence (intron 12) of NADH-GoGat gene on chromosome 3A (homeologous to NADH-GoGat gene on chromosome 3B) has been found statiscaly associated with yield, nitrogen uptake efficiency, grain weight and grain protein content in several field trials (2 years, 2 differents locations under several nitrogen conditions (optimal and sub-optimal).

The result of the allelic effect obtained by MLM statistical analysis on associated traits has shown that the allele comprising the sequence SEQ ID NO: 40 wherein the nucleotide at position 106 of said sequence is thymine, is the favourable allele for the yield and grain weight.

Accordingly, this association study in wheat shows the involvement of the NADH-GoGAT gene in NUE, yield and grain protein content in several nitrogen conditions (optimal and sub-optimal).

### EXAMPLE 4

### Wheat RNAi Transformation Constructs for NADH-GoGAT Repression

A 500 bp XbaI-XmnI synthetic fragment (represented as SEQ ID NO: 21) of wheat NADH-GoGAT is cloned in the pUC57 vector, leading to the pUC57_TaGOGAT vector. The XbaI-XmnI GOGAT RNAi fragment from pUC57-TaGOGAT is then introduced in the pENTR1A vector (Invitrogen) linearised with XbaI-XmnI, to create the pENTR1A_TaGOGAT.

An LR clonase reaction between the pENTR1A_TaGOGAT and the pAct-IR-66-SCV, allows the creation of pAct-TaGOGAT-RNAi-66-SCV (FIG. 3). pAct-IR-66-SCV is a vector used to create RNAi vectors under the control of the rice Actin gene promoter (McElroy et al., 1990). pAct-IR-66-SCV is obtained after introduction of the proActin-RNAi-terSac66 cassette from pBIOS890 into the binary vector pSCV1 (Firek et al., 1993). The binary plasmid pAct-TaGOGAT-RNAi-66-SCV is then introduced in the A. tumefaciens hypervirulent strain EHA105, and used for transformation experiments.

### EXAMPLE 5

### Experimental Validation of the NADH-GoGAT Gene Expression Between Arche and Recital

1) Materials & Methods

NADH-GOGAT gene expression has been analyzed for two bread wheat lines, i.e., Arche and Récital, using RT-PCR analysis with the following pair of primers: forward, AATTCTGGAAGGAAGGGCTTG; SEQ ID NO: 19; reverse: TTTGTATCCCTCGCGTATAGCTT; SEQ ID NO: 20. Samples (glumes, leave blades) were collected in Clermont-Ferrand (France) in 2008 under high N supply (240 kg N ha⁻¹ in four applications) and low N supply (40 kg N ha⁻¹ in one application). The experimental design was a split-plot with N treatment as the main plot and three replicates. Biological repetitions have been polled and RNA extracted.

2) Results

The results (see FIG. 4) show a significant difference in NADH-GoGAT expression between the two varieties under high nitrogen levels in the stems and leaves of the stage closest to the ear. In the rest of the plant and under low nitrogen levels, the level of NADH-GoGAT expression is very similar. These results support the hypothesis that NADH-GoGAT is a gene candidate for improving the grain filling of a wheat plant, beauce (1) there is a difference in GoGAT expression between the two varieties, (2) said difference appears under high level of nitrogen as appears the major QTL detected in the same genomic region, (3) the nitrogen assimilation mainly occurs in the upper leaves and stem segments.

### EXAMPLE 6

### Construction of a Transgenic Maize Plant Overexpressing a Wheat NADH-GoGAT

The maize cultivar A188 is transformed by the strain of Agrobacterium containing the vector-pSC4Act SynGOGAT TaMod-SCV described in Example 2 above, using the method described by Ishida et al., 1996 (cited above).

The genetically modified plant material (transformants) is selected as follows: the presence of the T-DNA and the number of copies of the transgene are determined by quantitative PCR (qPCR). In addition, the presence of the GFP reporter gene in both vectors used to obtain the transgenic plants allows sorting the transgenic seeds from the non-transgenic wild-type segregants.

The selected transformants is then regenerated into plants.

The transgenic plants are analyzed using routine methods: the number of copies of the integrated transgene and the integrity of the T-DNA. The full expression of the mRNA and the level of expression of the gene of interest are determined by quantitative PCR.

### REFERENCES

Agrama et al., (1999) Molecular Breeding 5:187-195.
An et al., (1986) Plant Physiology 81:86-91.
An et al., (2006) Plant and Soil 284:73-84.
Bechtold et al., (1993) C R Acad Sci III 316:1194-1199.
Bernard & Habash, (2009) New Phytologist 182:608-620.
Bertin & Gallais, (2001) Maydica 46:53-68.
Borrell et al., (1998) Australian Journal of Agricultural Research 49:829-843.
Cai et al., (2009) Plant Cell 28:527-537.
Caputo et al., (2009) Plant Physiol. Biochem. 47:335-342.
Chupeau et al., (1989) Biotechnology 7:503-508.
Depicker et al., (1982) J Mol. Appl. Genet. 1:561-73.
Finer et al., (1992) Plant Cell Report 11:323-328.
Firek et al., (1993) Plant Mol Biol. 22:129-142.
Fontaine et al., (2009) Theoretical and Applied Genetics 119:645-662.
Fromm et al., (1990) Nature 319:791-793.
Guerche et al., (1987) Biochimie 69:621-628.
Goldsbrough et al., (1993) Biotechnology 11:1286-1292.
Habash et al., (2007) Theoretical and Applied Genetics 114:403-419.
Hayakawa et al., (1994) Planta 193:455-460.
Hirel et al., (2001) Plant Physiology 125:1258-1270.
Ireland & Lea eds, (1999) The enzymes of glutamine, glutamate, asparagine and aspartate metabolism (Marcel Dekker, New York), pp 49-109.
Ishida et al., (1996) Nature Biotechnology 14:745-750.
Ishiyama et al., (2004) Journal of Biological Chemistry 279:16598-16605.
Jeanneau et al., (2002) Biochimie 84:1127-1135.
Kasuga et al., (1999) Nature Biotech. 17:287-291.
Katayama et al., (2000) Plant Mol. Biol. 44:99-106.
Kjaer et al., (1995) Genome 38:1098-1104.
Krapp et al., (2005) Photosynthesis Research 83:251-263.
Laperche et al., (2006) Theoretical and Applied Genetics 112:797-807.
Laperche et al., (2007) Theoretical and Applied Genetics 115:399-415.
Le Gouis et al., (2000) European Journal of Agronomy 12:163-173.
Lea & Miin, (2003) Plant Physiology and Biochemistry 41:555-564.
Lian et al., (2005) Theoretical and Applied Genetics 112:85-96.
Lima et al., (2006) Journal of Experimental Botany 57:2751-2761.
Loudet et al., (2003) Plant Physiology 131:1508-1508.
McCallum et al., (2000) Plant Physiology 123:439-442.
McElroy et al., (1990) Plant Cell 2:163-171.
McElroy et al., (1991) Mol. Gen. Genet. 231:150-160.
Miin & Habash, (2002) Journal of experimental botany 53:979-987.
Narusaka et al., (2003) Plant J. 34:137-148.
Noh and Amasino, (1999) Plant Mol. Biol. 41:181-194.
Obara, et al., (2001) Journal of Experimental Botany 52:1209-1217.
Ochs et al., (1999) Plant Molecular Biology 39:395-405.
Oury et al., (2003) Journal of Genetics & Breeding 57:59-68.
Potel et al., (2009) Febs Journal 276:4061-4076.
Rauh et al., (2002) Theoretical and Applied Genetics 104:743-750.
Simmonds, (1995) Journal of the Science of Food and Agriculture 67:309-315.
Tabuchi et al., (2007) Journal of Experimental Botany 58:2319-2327.
Thomas & Flavell, (1990) Plant Cell 2:1171-80.
Valadier et al., (2008) Febs Journal 275:3193-3206.
Verdaguer et al., (1996) Plant Mol. Biol. 6:1129-39.
Watson et al., (2005) FEBS Letters 579: 5982-5987.
Yamaya et al., (1992) Plant Physiology 100:1427-1432.
Yamaya et al., (2002) Journal of Experimental Botany 53:917-925.

1. A method for improving the grain filling of a plant, wherein said method comprises overexpressing in said plant a NADH-dependent glutamate synthase (NADH-GoGAT) having at least 95% identity with the polypeptide of sequence SEQ ID NO: 1.

2. The method of claim 1, wherein the grain filling is improved by increasing the grain weight and/or the grain protein content.

3. The method of claim 1, wherein said plant is a maize plant or a wheat plant.

4. The method of claim 1, wherein said NADH-GoGAT has the amino acid sequence SEQ ID NO: 22.

5. The method of claim 1, wherein said NADH-GoGAT is encoded by a nucleotide sequence selected from the group consisting of SEQ ID NO: 2, SEQ ID NO: 3, SEQ ID NO: 4, SEQ ID NO: 5, SEQ ID NO: 23, SEQ ID NO: 24, SEQ ID NO: 25 and SEQ ID NO: 26, preferably SEQ ID NO: 23, SEQ ID NO: 24, SEQ ID NO: 25 and SEQ ID NO: 26.

6. A recombinant expression cassette, wherein the cassette comprises a polynucleotide encoding a NADH-GoGAT as defined in claim 1, under control of a heterologous promoter functional in a plant cell.

7. A recombinant vector, wherein the vector contains an expression cassette comprising a polynucleotide encoding a NADH-GoGAT as defined in claim 1, under control of a promoter.

8. A host cell, wherein the host cell contains a recombinant expression cassette or a recombinant vector, wherein the recombinant expression cassette and recombinant vector each comprise a polynucleotide encoding a NADH-GoGAT as defined in claim 1.

9. A host cell of claim 8 which is a plant cell comprising a wheat plant cell or a maize plant cell.

10. A method for producing a transgenic plant, preferably a transgenic wheat plant or a transgenic maize plant, having an improved grain filling, wherein said method comprises:
providing a plant cell of claim 9;
regenerating from said plant cell a transgenic plant overexpressing a NADH-GoGAT having at least 95% identity with the polypeptide of sequence SEQ ID NO: 1.

11. A transgenic plant obtainable by the method of claim 10, said transgenic plant containing a recombinant expression cassette comprising said polynucleotide encoding a NADH-GoGAT.

12. A transgenic plant or an isolated organ or tissue thereof, wherein said transgenic plant or an isolated organ or tissue thereof comprises, stably integrated in its genome, a recombinant expression cassette comprising a polynucleotide encoding a NADH-GoGAT as defined in claim 1.

13. Seeds comprising wheat seeds or maize seeds comprised of a recombinant expression cassette comprising a polynucleotide encoding a NADH-GoGAT having at least 95% identity with the polypeptide of sequence SEQ ID NO: 1 obtained from a transgenic plant of claim 11.

14. An isolated wheat NADH-dependent glutamate synthase protein having at least 95% identity with the polypeptide of sequence SEQ ID NO: 1.

15. An isolated wheat NADH-dependent glutamate synthase protein of claim 14, wherein it has the amino acid sequence SEQ ID NO: 22.

16. An isolated polynucleotide chosen from the group consisting of:
a) a polynucleotide encoding a wheat NADH-GoGAT, which polypeptide has at least 95% identity with the polypeptide of sequence SEQ ID NO: 1;
b) a polynucleotide complementary to the polynucleotide a);
c) a polynucleotide capable of hybridizing selectively, under stringent conditions, with the polynucleotide a) or the polynucleotide b).

17. An isolated polynucleotide according to claim 16, wherein the isolated polynucleotide is selected from the group consisting of SEQ ID NO: 2, SEQ ID NO: 3, SEQ ID NO: 4, SEQ ID NO: 5, SEQ ID NO: 23, SEQ ID NO: 24, SEQ ID NO: 25 and SEQ ID NO: 26, preferably SEQ ID NO: 23, SEQ ID NO: 24, SEQ ID NO: 25 and SEQ ID NO: 26.

18. A pair of primers selected from the group consisting of the sequences SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, and SEQ ID NO: 19 and SEQ ID NO: 20, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, preferably SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34.

19. A method for identifying in a wheat plant (a) genetic marker(s) associated with an improved grain filling, wherein said method comprises genotyping said wheat plant and identifying one or more of the following alleles encoding a NADH-GoGAT as defined in claim 1:
an allele comprising the sequence SEQ ID NO: 35 wherein the nucleotide at position 109 of said sequence is guanine;
an allele comprising the sequence SEQ ID NO: 36;
an allele comprising the sequence SEQ ID NO: 37 wherein the nucleotide at position 133 of said sequence is adenine;
an allele comprising the sequence SEQ ID NO: 38 wherein the nucleotide at position 61 of said sequence is guanine;
an allele comprising the sequence SEQ ID NO: 39 wherein the nucleotide at position 439 of said sequence is guanine; and
an allele comprising the sequence SEQ ID NO: 40 wherein the nucleotide at position 106 of said sequence is thymine.

20. A method for selecting a wheat plant having an improved grain filling, wherein said method comprises identifying in wheat plants to be tested (a) genetic marker(s) associated with an improved grain filling by the method of claim 19, and selecting a plant containing said genetic marker(s).
